(19) United States
(12) Patent Application Publication
(10) Pub. No.: US 2008/0293720 A1
(43) Pub. Date: Nov. 27, 2008

(54) Pyridinyl Sulfonamide Modulators of Chemokine Receptors

(75) Inventors: Pamela A. Cleary, King of Prussia, PA (US); Krista B. Goodman, King of Prussia, PA (US); Simon Peace, Hertfordshire (GB); Joanne Philp, King of Prussia, PA (US); Clark A. Sehon, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited, Greenford Middlesex (GB)

(21) Appl. No.: 12/096,185

(22) PCT Filed: Dec. 4, 2006
(86) PCT No.: PCT/US06/61543

§ 371 Date: Jun. 5, 2008

(30) Foreign Application Priority Data
Dec. 5, 2005 (GB) 0524786.1

(51) Int. Cl. A61K 31/44; C07D 213/30; A61K 31/4439; C07D 417/02; C07D 401/02; A61K 31/4545; A61K 31/496; A61K 31/5377; C07D 413/02

(52) U.S. Cl. 514/237.2; 514/347; 546/294; 514/342; 546/270.7; 546/194; 514/318; 514/317; 544/360; 514/253.12; 544/131

(57) ABSTRACT

The present invention relates to compounds of following formula:

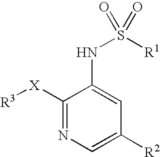

or pharmaceutically acceptable salts thereof; pharmaceutical compositions containing them, and their use in the treatment of disorders mediated by the CCR-2 receptor.

The present invention relates to a class of pyridinyl sulfonamides that are modulators of chemokine receptors, particularly as CCR2 antagonists and their methods of use.

CCR2 is a chemokine receptor that is expressed on a cell surface of monocycles and some other blood leukocytes. CCR2 binds to the monocyte chemotactic protein MOP-1, and other CC chemokines, which are produced at sites of inflammation and infection. Recruitment of monocytes to inflammatory sites by MCP-1/CCR2 interactions has been implicated in driving the pathogenesis of a number of diseases including multiple inflammatory disorders including rheumatoid arthritis, atherosclerosis, multiple sclerosis, bronchiolitis obliterans syndrome, asthma, allergic rhinitis, eczema, atopic dermatitis, kidney disease, alveolitis, nephritis, liver cirrhosis, congestive heart failure, viral meningitis, cerebral infarction, neuropathy, Kawasaki disease, Alzheimer's disease, stroke, acute nerve injury, HIV infection, AIDS, autoimmune diseases, cancer, sepsis, retinosis, inflammatory bowel disease, transplant arteriosclerosis, idiopathic pulmonary fibrosis, psoriasis, HIV-associated dementia, lupus, erthematosis, hepatitis, pancreatitis, Crohn's disease, endometriosis, and diabetes.

Accordingly, it would be an advance in the art to discover a class of compounds that bind to CCR2, thereby preventing or minimizing the formation of the undesirable MCP1-mediated recruitment of monocytes to inflammatory sites.

### SUMMARY OF THE INVENTION

In a first aspect of the invention, there is provided a compound of the following formula:

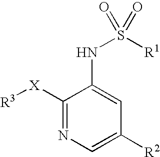

or a pharmaceutically acceptable salt thereof;
wherein:
X represents —O, —NH or —S;
R¹ is a heteroaryl group or an aryl group, each substituted with up to three substituents independently selected from the group consisting of halo, hydroxy, C₁₋₆ alkyl, C₁₋₄ alkoxy, hydroxy-C₁₋₄ alkyl-, C₁₋₄ alkoxy-C₁₋₄ alkyl-, C₃₋₆ cycloalkyl, —CN, C₁₋₄ alkylthio-, —OCF₃, dimethylamino, nitro, and —CF₃;
R² is H, C₁₋₆ alkyl, C₁₋₆ alkoxy, halo, —CN, —CF₃, or —OCF₃;
R³ is R⁴-phenyl-, R⁵-pyridyl-, methyltetrazolyl, morpholino-C(O)—CH₂—; (CH₃)₂N(CH₂)₂N(CH₃)—C(O)CH₂—, methylisoxazolyl; pyridazinyl, or triazolyl;
where R⁴ is H, halo, CN, hydroxymethyl, tetrazolyl, COOH, or —CR^{a}R^{b}—NR^{c}R^{d};
R⁵ is 2-methyl, 2-halo, 2-cyano, 2-COOH, dimethylaminomethyl, methylmorpholinomethyl;
where R^{a} and R^{b} are each H or, together with the C atom to which they are attached, form a carbonyl group;
R^{c} is H, —(CH₂)_{y}—R⁶; C₃-C₆-cycloalkyl; C₁-C₆-alkyl; phenyl-CH(CH₃)—; oxoisoxazolidinyl; dimethylthiazolyl; dihydrothiazolyl; imidazolyl-CH₂CH(CH₂OH)—CH₂—; or imidazolyl-CH₂CH(CH₂OH)—;
R^{d} is H, C₁-C₄-alkyl, benzyl or, R^{c} and R^{d}, together with the nitrogen to which they are attached form a piperidinyl, pyrrolidinyl, difluoropyrrolidinyl, morpholino, pyrazinyl, triazolopiperidinyl, or pyrrolidinonyl group;
y is 1, 2, or 3;
R⁶=—NR⁷R⁸, OH, methoxy, phenyl, imidazolyl, indolyl, tetrahydropyranyl, benzimidazolyl, or C₃-C₆-cycloalkyl;
R⁷ is H or methyl; R⁸ is H, C₁-C₄-alkyl, or phenyl; or R⁷ and R⁸, together with the nitrogen to which they are attached form a piperidinyl, pyrrolidinyl, morpholino, pyrazinyl, imidazolyl, or pyrrolidinonyl group.

In a second aspect, the present invention is a composition that comprises a) the compound of Claim 1 or a pharmaceutically acceptable salt thereof; and b) a pharmaceutically acceptable excipient.

In a third aspect, the invention provides method of treating a disease or condition mediated by CCR2 comprising administering to a patient in need thereof a pharmaceutically effective amount of the compound of the present invention or a pharmaceutically acceptable salt thereof.

### DETAILED DESCRIPTION OF THE INVENTION

In a first aspect, the present invention relates to a compound of the following formula:

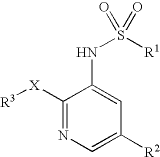

or a pharmaceutically acceptable salt thereof;
wherein:
X represents —O, —NH or —S;
R¹ is a heteroaryl group or an aryl group, each substituted with up to three substituents independently selected from the group consisting of halo, hydroxy, C₁₋₆ alkyl, C₁₋₄ alkoxy, hydroxy-C₁₋₄ alkyl-, C₁₋₄ alkoxy-C₁₋₄ alkyl-, C₃₋₆ cycloalkyl, —CN, C₁₋₄ alkylthio-, —OCF₃, dimethylamino, nitro, and —CF₃;
R² is H, C₁₋₆ alkyl, C₁₋₆ alkoxy, halo, —CN, —CF₃, or —OCF₃;
R³ is R⁴-phenyl-, R⁵-pyridyl-, methyltetrazolyl, morpholino-C(O)—CH₂—; (CH₃)₂N(CH₂)₂N(CH₃)—C(O)CH₂—, methylisoxazolyi; pyridazinyl, or triazolyl;
where R⁴ is H, halo, CN, hydroxymethyl, tetrazolyl, COOH, or —CR^{a}R^{b}—NR^{c}R^{d};
R⁵ is 2-methyl, 2-halo, 2-cyano, 2-COOH, dimethylaminomethyl, methylmorpholinomethyl;
where R^{a} and R^{b} are each H or, together with the C atom to which they are attached, form a carbonyl group;
R^{c} is H, —(CH₂)_{y}—R⁶; C₃-C₆-cycloalkyl; C₁-C₆-alkyl; phenyl-CH(CH₃)—; oxoisoxazolidinyl; dimethylthiazolyl; dihydrothiazolyl; imidazolyl-CH₂CH(CH₂OH)—CH₂—; or imidazolyl-CH₂CH(CH₂OH)—;
R^{d} is H, C₁-C₄-alkyl, benzyl or, R^{c} and R^{d}, together with the nitrogen to which they are attached form a piperidinyl, pyrrolidinyl, difluoropyrrolidinyl, morpholino, pyrazinyl, triazolopiperidinyl, or pyrrolidinonyl group;
y is 1, 2, or 3;
R⁶=—NR⁷R⁸, OH, methoxy, phenyl, imidazolyl, indolyl, tetrahydropyranyl, benzimidazolyl, or C₃-C₆-cycloalkyl;
R⁷ is H or methyl; R⁸ is H, C₁-C₄-alkyl, or phenyl; or R⁷ and R⁸, together with the nitrogen to which they are attached form a piperidinyl, pyrrolidinyl, morpholino, pyrazinyl, imidazolyl, or pyrrolidinonyl group.

As used herein, the term “alkyl” refers to straight or branched hydrocarbon chains containing the specified number of carbon atoms. Examples of “alkyl” as used herein include, but are not limited to, methyl, ethyl, n-propyl, n-butyl, n-pentyl, n-hexyl, isobutyl, isopropyl, t-butyl, and 1,1-dimethylpropyl.

Examples of “alkoxy” include, but are not limited to methoxy, ethoxy, n-propoxy, prop-2-oxy, n-butoxy, but-2-oxy, 2-methylprop-1-oxy, 2-methylprop-2-oxy, n-pentoxy and n-hexyloxy.

Examples of C₁₋₄ alkylthio include, but are not limited to methylthio, ethylthio, n-propylthio, prop-2-thio, n-butylthio, but-2-thio, 2-methylprop-1-thio, 2-methylprop-2-thio.

Examples of C₃₋₆ cycloalkyl groups include cyclopropyl, cyclobutyl, cyclopentyl, and cyclohexyl groups.

As used herein, the term “heterocycloalkyl” refers to a 5-6 membered non-aromatic ring group containing one or more heteroatoms. Examples of heterocycloalkyl groups include 2H-pyrrolyl, pyrrolinyl, pyrrolidinyl, imidazolinyl, imidazolidinyl, oxazolinyl, oxazolidinyl, pyrazolinyl, pyrazolidinyl, 2H-pyridinyl, morpholinyl, thiomorpholinyl, dihydropyridazinyl, piperidinyl, and piperazinyl.

The term “halogen” or “halo” refers to fluoro, chloro, bromo or iodo.

The terms “a compound of the present invention” and “the compound of the present invention” are used herein to refer to one or more compounds of the present invention. The present invention includes compounds as well as their pharmaceutically acceptable salts. Accordingly, the word “or” in the context of a compound or a pharmaceutically acceptable salt thereof, is understood to include a compound or a pharmaceutically acceptable salt thereof or a combination thereof.

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio. The skilled artisan will appreciate that pharmaceutically acceptable salts of the compounds according to Formula (I) may be prepared. These pharmaceutically acceptable salts may be prepared in situ during the final isolation and purification of the compound, or by separately reacting the purified compound in its free acid or free base form with a suitable base or acid, respectively.

Compounds of the invention may be prepared by processes in accordance with the following reaction schemes.

Compounds of formula (I) may be prepared by the reaction of a compound of formula (II)

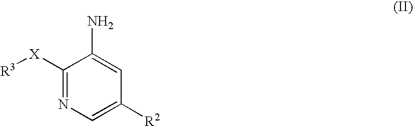

or an appropriately protected derivative thereof wherein R², R³ and X are as defined above, with a compound of formula (III)

L-SO₂—R¹ (III)

wherein L is a suitable leaving group, and R¹ is as defined above;
and thereafter optionally,
(i) removing any protecting group(s); and/or
(ii) forming a salt; and/or
(iii) converting a compound of formula (I) or a salt thereof to another compound of formula
(I) or a salt thereof.

Examples of suitable leaving groups L include chloro, bromo, and pentafluorophenoxy.

Typically, where L represents chloro, such a reaction may be carried out by dissolving the compound of formula (II) in a suitable solvent, for example pyridine optionally mixed with a second solvent, such as chloroform or tetrahydrofuran, and reacting it with the compound of formula (III) also in a suitable solvent, for example pyridine. The addition of a catalytic quantity of dimethylaminopyridine may also be used. The reaction would generally be carried out at elevated temperature in the region of 80-250° C., for example at about 200° C., for a period of 30 minutes to 1 hour or at 80° C. for a period of 5-24 hours.

Compounds of the formula (II) may be prepared according to the chemistry detailed in scheme 1 below, for example by treatment of alcohols, amines or thiols bearing the relevant group R³ with an appropriate base, for example, a compound of formula (IV), potassium carbonate, and a nitro-pyridine bearing an appropriate leaving group L, such as chlorine, in a solvent such as dimethylformamide (DMF) for an appropriate length of time, for example 1-24 hours, at a temperature from 20-100° C. The nitro-pyridine product (III) of this reaction may then be reduced using standard literature techniques; for example, hydrogenation in the presence of a metal catalyst such as platinum or palladium. Alternatively transfer hydrogenation with an appropriate reductant, for example ammonium formate, may be performed in the presence of a palladium or platinum catalyst in an appropriate solvent, for example dichloromethane, ethyl acetate, ethanol or a mixed solvent system to provide a compound of formula (IIa).

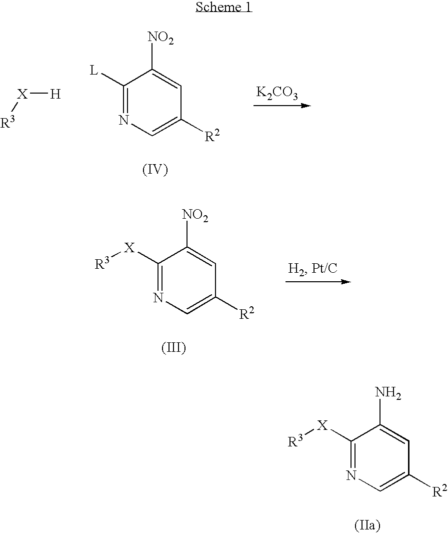

Nitro-pyridine compounds of formula (IV) can be readily prepared by a person skilled in the art using literature methods. An example of a method of preparing compounds of formula (IV) wherein R² represent CN is given in scheme 5 below.

More specifically compounds of formula (I) wherein X represents O and R³ is, for example a 6-membered aromatic or heteroaromatic ring bearing a carboxylic acid substituent can be prepared as shown in scheme 2 below by hydrolysis of a corresponding methyl or ethyl ester.

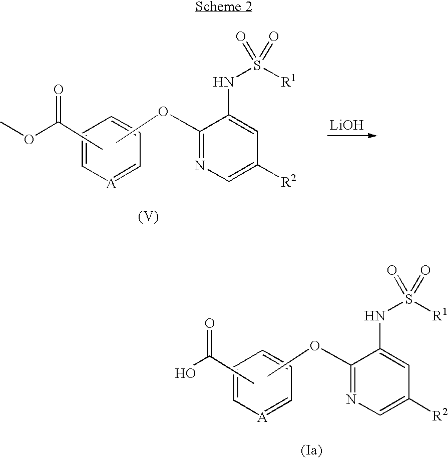

wherein A represents CH₂ or a heteroatom such as O, N or S. Suitable hydrolysis conditions include, for example lithium hydroxide in a suitable solvent such as methanol. This reaction may be carried out at 50-90° C. until complete, such as for 15 minutes to 24 hours.

Compounds of formula (I) wherein X represents O and R³ is phenyl bearing an amide substituent can be prepared according to the chemistry described in scheme 3 below.

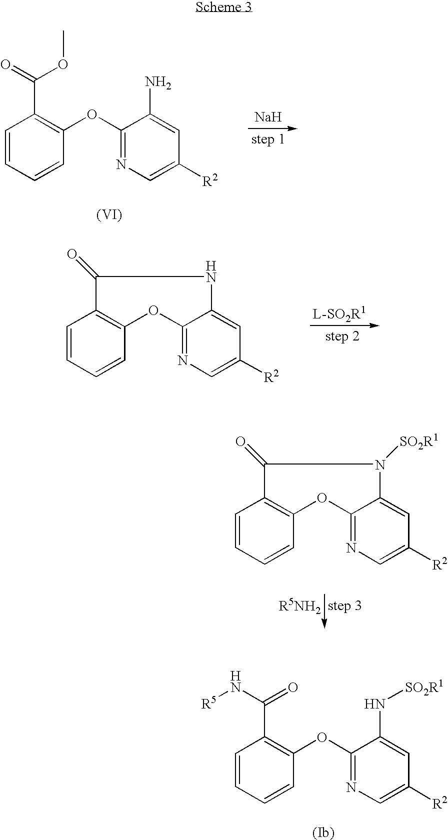

Step 1, cyclisation of an appropriate ester with a base, for example potassium sodium hydride, can be effected in an appropriate solvent such as tetrahydrofuran, at, for example 0° C. The intermediate lactam may be isolated or immediately treated with a sulfonylating reagent, as shown in step 2. The resulting sulfonylated lactam may then be opened as shown in step 3 to provide the amide by treatment with the relevant amine.

Alternatively, compounds of the general formula (Ib) may be prepared from the corresponding acid by treatment with the relevant amine in the presence of an amide coupling reagent, for example TBTU, according to standard literature procedures as shown in scheme 4 below.

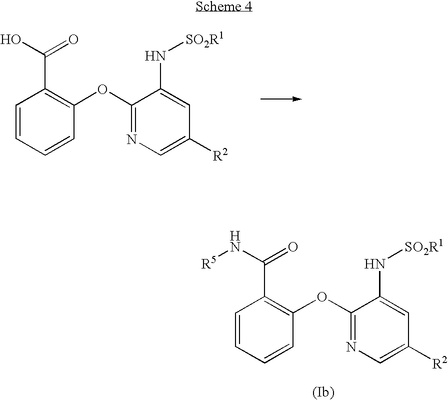

2-Chloro-3-nitropyridines (compound IVa) may be prepared by chlorination/dehydration of the corresponding 2-hydroxy pyridines as shown in Scheme 5 below. Compounds of formula (VII) may be dehydrated to provide the corresponding chloro/cyano substituted compound.

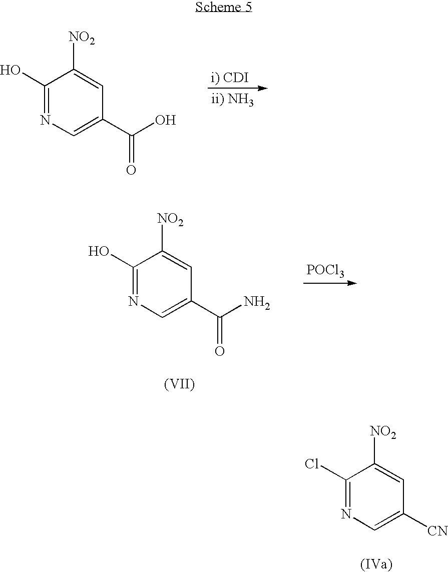

Intermediate (V) as shown above in scheme 2, where R¹ represents aryl, may be prepared by, for example the method set out in scheme 6 below:

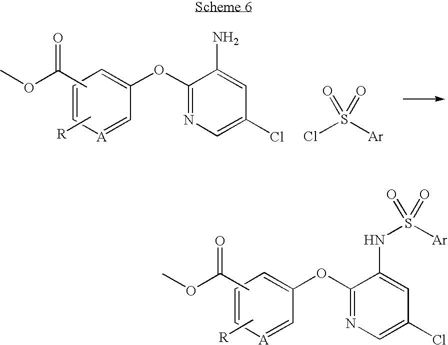

Wherein A represents CH₂ or a heteroatom such as O, N or S and R represents an appropriate substituent within the definitions defined for compounds of formula (I). The reaction may be effected in solvent such as pyridine at an elevated temperature, for example 50 to 100° C. such as 95° C. for approximately 1 hour.

Intermediate (VI) shown in scheme 3 above may be prepared as shown in scheme 7 below:

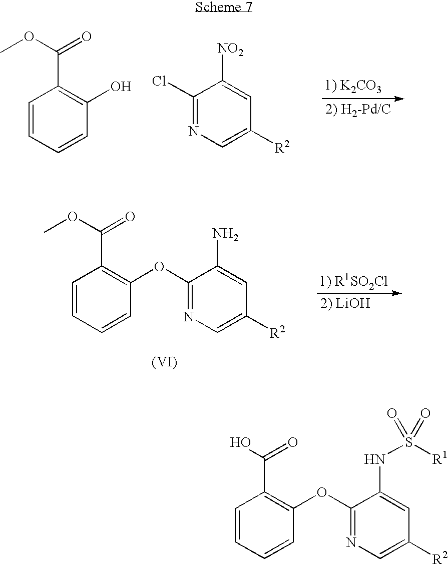

Treatment with potassium carbonated my be effected in a suitable solvent such as DMF at an elevated temperature, for example in the range 80 to 120° C. such as 100° C. for up to 8 hours, such as 4 hours.

Hydrogenation in the presence of a catalyst such as palladium on carbon may be effected in a suitable solvent such as ethyl acetate at room temperature, for up to 4 hours such as 2 hours.

The coupling step between the amine and the sulfonyl chloride group in Scheme 7 above may be effected by, for example, refluxing the starting materials in a solvent such as methanol and water in the presence of lithium hydroxide for a suitable period such as 1 hour.

Those skilled in the art will appreciate that in the preparation of the compounds of the invention or a salt or solvate thereof it may be necessary and/or desirable to protect one or more sensitive groups in the molecule to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl type protecting groups (e.g. benzyl, trityl, chlorotrityl). Examples of suitable oxygen protecting groups may include for example alkyl silyl groups, such as trimethylsilyl or tert-butyldimethylsilyl; alkyl ethers such as tetrahydropyranyl or tert-butyl; or esters such as acetate.

Other compounds of formula (I) may be prepared by general methods analogous to those described above.

The compounds of formula (I) as defined above contain a basic grouping and may also contain an acidic grouping and therefore may form salts with physiologically acceptable acids or bases.

Examples of pharmaceutically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-physiologically acceptable anion or cation are within the scope of the invention as useful intermediates for the preparation of physiologically acceptable salts and/or for use in non-therapeutic, for example, in vitro, situations.

Certain of the compounds of the invention may form acid addition salts with one or more equivalents of the acid. Certain of the compounds of the invention may form acid addition salts with less than one equivalent of the acid. The present invention includes within its scope all possible stoichiometric and non-stoichiometric forms.

Certain compounds of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. Likewise, it is understood that compounds of formula (I) may exist in tautomeric forms other than that shown in the formula and these are also included within the scope of the present invention.

When a specific enantiomer of a compound of general formula (I) is required, this may be obtained for example by resolution of a corresponding enantiomeric mixture of a compound of formula (I) using conventional methods. Thus the required enantiomer may be obtained from the racemic compound of formula (I) by use of chiral HPLC procedure.

According to a further aspect, the invention provides method of treating a disease mediated by CCR2 comprising administering the compound of formula (I), or a pharmaceutically acceptable salt thereof to a patient in need thereof.

It will be appreciated by those skilled in the art that reference herein to treatment extends to prophylaxis as well as the treatment of established diseases or symptoms.

While it is possible that, for use in therapy, a compound of the invention may be administered as the raw chemical, it is preferable to present the active ingredient as a pharmaceutical formulation.

The invention thus further provides a pharmaceutical composition comprising a compound of formula (I) or a pharmaceutically acceptable salt thereof together with one or more pharmaceutically acceptable carriers or diluents. The carrier(s), diluent(s) and/or excipient(s) must be ‘acceptable’ in the sense of being compatible with the other ingredients of the formulation and not deleterious to the recipient thereof.

The compounds of the invention may be administered in conventional dosage forms prepared by combining a compound of the invention with standard pharmaceutical carriers, diluents or excipients according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, buccal, topical, inhalation or insufflation, implant, rectal or parenteral administration to mammals including humans.

The compositions may be in the form of tablets, capsules, powders, granules, lozenges, creams or liquid preparations, such as oral or sterile parenteral solutions or suspensions.

Tablets and capsules for oral administration may contain conventional excipients such as binding agents, for example, syrup, acacia, gelatin, sorbitol, tragacanth, mucilage of starch or polyvinylpyrrolidone; fillers, for example, lactose, sugar, microcystalline cellulose, maize-starch, calcium phosphate, glycine or sorbitol; lubricants, for example, magnesium stearate, stearic acid, talc, polyethylene glycol or silica; disintegrants, for example, potato starch or sodium starch glycollate, or wetting agents such as sodium lauryl sulphate. The tablets may be coated according to methods well known in the art. Oral liquid preparations may be in the form of, for example, aqueous or oily suspensions, solutions emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxyethylcellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example, almond oil, fractionated coconut oil, oily esters, propylene glycol or ethyl alcohol; solubilizers such as surfactants for example polysorbates or other agents such as cyclodextrins; and preservatives, for example, methyl or propyl p-hydroxybenzoates or ascorbic acid; and, if desired, conventional flavouring or colouring agents. The compositions may also be formulated as suppositories, e.g. containing conventional suppository bases such as cocoa butter or other glycerides.

For buccal administration the composition may take the form of tablets or lozenges formulated in conventional manner.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

The composition according to the invention may be formulated for parenteral administration by injection or continuous infusion. Formulations for injection may be presented in unit dose form in ampoules, or in multi-dose containers with an added preservative. The compositions may take such forms as suspensions, solutions, or emulsions in oily or aqueous vehicles, and may contain formulatory agents such as suspending, stabilising and/or dispersing agents. Alternatively the active ingredient may be in powder form for constitution with a suitable vehicle, e.g. sterile, pyrogen-free water, before use.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

Where the compositions comprise dosage units, each unit will typically contain from 1-1000 mg of the active ingredient.

Further details for the preparation of compounds of formula (I) are found in the Examples section hereinafter.

The following non-limiting examples illustrate the present invention.

### Abbreviations

HPLC—high-performance liquid chromatography
DCM—dichloromethane
DMF—dimethylformamide
DMSO—dimethylsulfoxide
HCl—hydrochloric acid
SPE—solid phase extractor
H BtU—O-Benzotriazol-1-yl-N,N,N′,N′-tetramethyluronium tetrafluoroborate

### DMAP—N,N-dimethylaminopiperidine

MDAP—mass directed autopreparative HPLC

Mass spectra were obtained using either a Waters ZQ mass spectrometer or Micromass Platform 2 mass spectrometer and use electro-spray ionisoation to observe either MH+ or M−. Proton Nuclear Magnetic Resonance (¹H-NMR) spectra were recorded at 400 MHz unless otherwise stated, chemical shifts are reported in ppm downfield from Me₄Si, used as internal standard, and are assigned as singlets (s), doublets (d), doublets of doublets (dd), triplets (t), doublet of triplets (dt), quartets (q) multiplets (m) or are otherwise described in full. The prefix “br” refers to a broad peak; for example, a broad single may appear as br.s (or br s).

### Intermediate 1 5-nitro-6-oxo-1,6-dihydro-3-pyridinecarboxamide

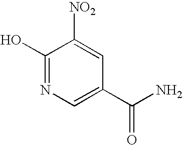

Carbonyl diimidazole (CDI, 103 g) was added portion-wise to a solution of 5-nitro-6-oxo-1,6-dihydro-3-pyridinecarboxylic acid (106 g) in DMF (510 mL) at room temperature and the mixture heated to 60° C. until bubbling ceased. The mixture was cooled to room temperature and poured into 10-35% aqueous ammonia (730 mL). After 20 minutes the precipitate was filtered and washed with water (50 mL) and dried in vacuo at 70° C. to yield 83.3 g of the product as a bright yellow solid; ¹H NMR (d₆-DMSO) δ 8.61 (d, 1H), 8.55 (d, 1H), 7.80 (br.s, 1H), 7.17 (br.s, 1H).

### Intermediate 2 6-chloro-5-nitro-3-pyridinecarbonitrile

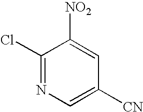

Intermediate 1 (10 g) in phosphorous oxychloride (100 mL) was refluxed for 3 hours followed by vigorously refluxing for a further 3 hours and cooling to room temperature overnight. The mixture was concentrated, poured onto ice (300 mL) and stirred for 15 minutes. The resulting solid was filtered and triturated with hot ethyl acetate (250 mL), filtered hot and the organic filtrate washed with water (3×150 mL), dried over magnesium sulfate and concentrated to give the title compound as 5.94 g of a light yellow powder; ¹H NMR (d₆-DMSO) δ 8.88 (d, 1H), 8.50 (d, 1H).

### Intermediate 3 2,5-dichloro-3-nitropyridine

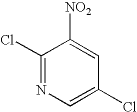

5-Chloro-3-nitro-2-pyridinamine (5.0 g, 28.8 mmol) was dissolved in conc. HCl (50 mL) and cooled to −10° C. in an ice-salt bath. Sodium nitrite (4.97 g, 72 mmol) in water (10 mL) was added dropwise to the cooled solution over 1 h and stirred at 0° C. for 1 h longer. The reaction mixture was cooled to −10° C. in an ice-salt bath and neutralized with 2N sodium hydroxide to pH 9.0 keeping the temperature below 0° C. EtOAc (150 mL) was added and the mixture was filtered. The organic layer was separated, dried over sodium sulfate and concentrated in vacuo to afford the title compound as 3.36 g (60% yield) of a light brown solid; ¹H NMR (d₆-DMSO) δ 8.62 (d, 1H, 2.5 Hz), 8.26 (d, 1H, 2.2 Hz).

### Intermediate 4 methyl 2-[(5-chloro-3-nitro-2-pyridinyl)oxy]benzoate

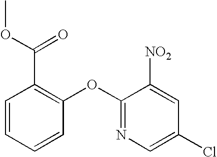

To a stirred solution of the 2,5-dichloro-3-nitropyridine (2 g) and methyl 2-hydroxybenzoate (1.4 ml) in DMF (10 ml) was added potassium carbonate. The resultant mixture was heated to 50° C. for 24 hours. The crude reaction was cooled and partitioned between ethyl acetate and water (75 ml each), separated and the aqueous extracted with ethyl acetate (225 ml total). The combined organic layers were washed with water, brine, dried over sodium sulfate and concentrated in vacuo to afford 3.24 g of the title compound; MH+ 309.

### Intermediate 5 5-chloro-2-[(4-methyl-2-pyridinyl)oxy]-3-nitropyridine

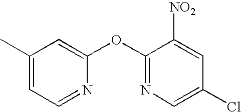

To a solution of 2,5-dichloro-3-nitropyridine (386 mg) and 4-methyl-2(1H)-pyridinone (241 mg) in DMF (10 ml) was added potassium carbonate (552 mg). The resultant mixture was heated to 50° C. for 1½ h then at 70° C. for 2 h before been cooled to room temperature and stirred for 72 h. The mixture was concentrated to give a solid which was partitioned between DCM/water, the layers were separated and water layer extracted with further DCM. The combined DCM extracts were washed (water), dried over sodium sulfate and concentrated in vacuo to afford a brown oil. The oil was purified by silica gel SPE (20-40% ethyl acetate in cyclohexane) to afford 197 mg of the title compound; MH+ 266.

### Intermediate 6 5-chloro-2-[(2-methyl-3-pyridinyl)oxy]-3-nitropyridine

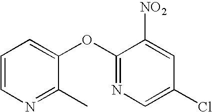

A solution of 2,5-dichloro-3-nitropyridine (1.93 g) in DMF (10 mL) was prepared and 2 mL of this solution added to 2-methyl-3-hyroxypyridine (229 mg). Potassium carbonate (1.5 eq) was added and the mixture heated to 90° C. for one hour. The mixture was then cooled and concentrated, dissolved in DCM and concentrated a second time. The resulting solid was partitioned between chloroform and water (5 mL each) and separated.

The chloroform layer was blown down to provide the crude product. ¹H NMR (CDCl₃) δ 8.47 (dd, 1H), 8.38 (d, 1H), 8.26 (d, 1H), 7.43 (dd, 1H), 7.23 (dd, 1H), 2.47 (s, 3H).

The following intermediates were prepared employing a similar method to that described for intermediate 5 above. The reaction may be run from 20-90° C. for 1-24 hours. Ethyl acetate may be used instead of chloroform during the work-up.

| Intermediate | | | | |
|---|---|---|---|---|
| No. | Precursor(s) | Structure | Data | Name |
| 7 | 3-fluoro pyridine& 2,5-dichloro-3-nitropyridine | 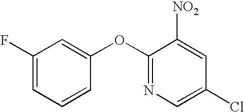 | RT;3.4 mins | 5-chloro-2-[(3-fluorophenyl)oxy]-3-nitropyridine |
| 8 | 3-chloro-5-hydroxypyridine& 2,5-dichloro-3-nitropyridine | 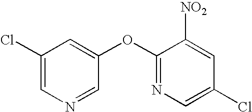 | MH⁺; 286 | 5-chloro-2-[(5-chloro-3-pyridinyl)oxy]-3-nitropyridine |
| 9 | methyl 5-hydroxy-3-pyridinecarboxylate& 2,5-dichloro-3-nitropyridine | 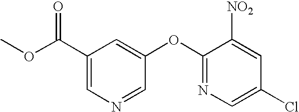 | MH⁺; 310 | Methyl-5-[(5-chloro-3-nitro-2-pyridinyl)oxy]-3-pyridinecarboxylate |
| 10 | methyl 2-fluoro-6-hydroxybenzoate& 2,5-dichloro-3-nitropyridine | 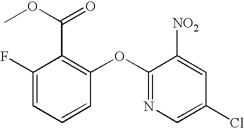 | MH⁺; 327 | methyl 2-[(5-chloro-3-nitro-2-pyridinyl)oxy]-6-fluorobenzoate |
| 11 | 3-hydroxypyridine& 2,5-dichloro-3-nitropyridine | 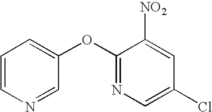 | MH⁺; 252 | 5-chloro-3-nitro-2-(3-pyridinyloxy)pyridine |
| 12 | methyl 3-hydroxybenzoate& 2,5-dichloro-3-nitropyridine | 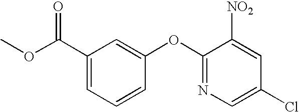 | MH⁺; 309 | Methyl-3-[(5-chloro-3-nitro-2-pyridinyl)oxy]benzoate |
| 13 | methyl 4-hydroxybenzoate& 2,5-dichloro-3-nitropyridine | 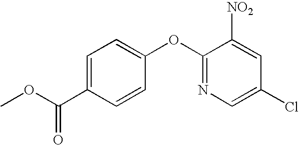 | MH⁺; 309 | Methyl-4-[(5-chloro-3-nitro-2-pyridinyl)oxy]benzoate |
| 14 | 2-methyl-3-hydroxypyridine &6-chloro-5-nitro-3-pyridinecarbonitrile | 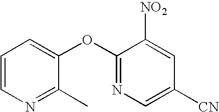 | MH⁺; 257 | 6-[(2-methyl-3-pyridinyl)oxy]-5-nitro-3-pyridinecarbonitrile |
| 15 | 6-methyl-3-hydroxypyridine &6-chloro-5-nitro-3-pyridinecarbonitrile | 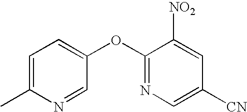 | MH⁺; 257 | 6-[(6-methyl-3-pyridinyl)oxy]-5-nitro-3-pyridinecarbonitrile |
| 16 | 3(2H)-pyridazinone& 2,5-dichloro-3-nitropyridine | 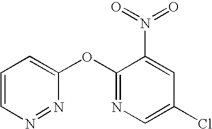 | MH⁺; 302 | 3-[(5-chloro-3-nitro-2-pyridinyl)oxy]pyridazine |
| 17 | 2,5-dichloro-3-nitropyridine2,6-dimethyl-3-pyridinol | 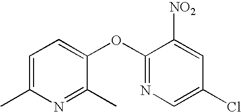 | MH⁺; 282 | 5-chloro-2-[(2,6-dimethyl-3-pyridinyl)oxy]-3-nitropyridine |
| 18 | 2,5-dichloro-3-nitropyridine5-methyl-3(2H)-isoxazolone | 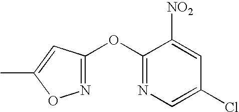 | MH⁺; 256 | 5-chloro-2-[(5-methyl-3-isoxazolyl)oxy]-3-nitropyridine |
| 19 | 2-[(dimethylamino)methyl]-3-pyridinol &2,5-dichloro-3-nitropyridine | 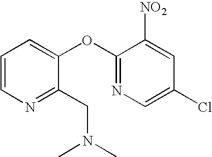 | MH⁺; 310 | ({3-[(5-chloro-3-nitro-2-pyridinyl)oxy]-2-pyridinyl}methyl)dimethylamine |
| 20 | 2-chloro-3-pyridinol& 2,5-dichloro-3-nitropyridine | 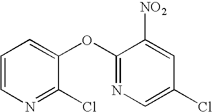 | MH⁺; 286 | 5-chloro-2-[(2-chloro-3-pyridinyl)oxy]-3-nitropyridine |
| 21 | 3-hydroxy-2-pyridinecarbonitrile& 2,5-dichloro-3-nitropyridine | 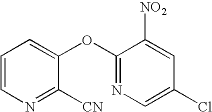 | M⁺; 277 | 3-[(5-chloro-3-nitro-2-pyridinyl)oxy]-2-pyridinecarbonitrile |
| 22 | 2-chlorophenol &2,5-dichloro-3-nitropyridine | 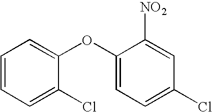 | MH⁺; 285 | 4-chloro-1-[(2-chlorophenyl)oxy]-2-nitrobenzene |
| 23 | 3-hydroxybenzonitrile& 2,5-dichloro-3-nitropyridine | 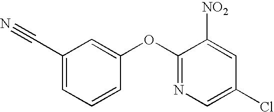 | MH⁺; 276 | 3-[(5-chloro-3-nitro-2-pyridinyl)oxy]benzonitrile |
| 24 | 4-hydroxybenzonitrile& 2,5-dichloro-3-nitropyridine | 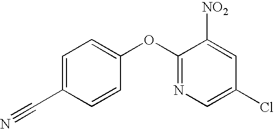 | MH⁺; 276 | 4-[(5-chloro-3-nitro-2-pyridinyl)oxy]benzonitrile |
| 25 | Phenol& 2,5-dichloro-3-nitropyridine | 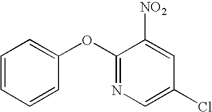 | MH⁺; 251 | 5-chloro-3-nitro-2-(phenyloxy)pyridine |
| 26 | 2,5-dichloro-3-nitropyridine & 2-(4-morpholinyl)-2-oxoethanol | 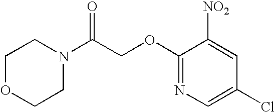 | MH⁺; 302 | 4-{[(5-chloro-3-nitro-2-pyridinyl)oxy]acetyl}morpholine |
| 27 | 2,5-dichloro-3-nitropyridine & N-[2-(dimethylamino)ethyl]-2-hydroxy-N-methylacetamide | 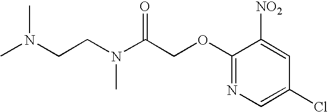 | MH⁺; 317 | 2-[(5-chloro-3-nitro-2-pyridinyl)oxy]-N-[2-(dimethylamino)ethyl]-N-methylacetamide |
| 28 | 2,5-dichloro-3-nitropyridine & 1-methyl-1,2-dihydro-5H-tetrazol-5-one | 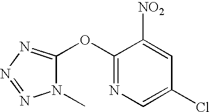 | MH⁺; 257 | 5-chloro-2-[(1-methyl-1H-tetrazol-5-yl)oxy]-3-nitropyridine |

### Intermediate 29 methyl 2-[(3-amino-5-chloro-2-pyridinyl)oxy]benzoate

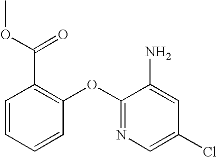

To a stirred solution of methyl 2-[(5-chloro-3-nitro-2-pyridinyl)oxy]benzoate (intermediate 4) (997 mg) in DCM (10 ml) was added ammonium formate (3 g) and 5% platinum on carbon “degussa” (252 mg). The resultant mixture was heated to 50° C. for 24 hours. The crude mixture was cooled, filtered through celite and concentrated in vacuo to afford the title compound, 873 mg (97% yield); MH+ 279/281.

### Intermediate 30 methyl 2-[(3-amino-2-pyridinyl) oxy]benzoate

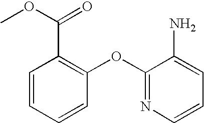

2-Chloro-3-nitropyridine (10 g), methyl 2-hydroxybenzoate (11.5 g) and potassium carbonate (17.4 g) were mixed in DMF (100 mL) and heated to 100° C. for 4 hours. The mixture was allowed to cool to room temperature and was then poured into water (600 mL) and extracted with ethyl acetate (2×150 mL). The combined organics was washed with water (100 mL) and brine (200 mL) and concentrated in vacuo. The resulting solid was triturated with hexane and filtered. The resulting solid (1 g) was hydrogenated for 2 hours in ethyl acetate (40 mL) and ethanol (10 mL) in the presence of 5% palladium on carbon (100 mg). Filtration through celite provided 0.76 g of the title compound; ¹H NMR (CDCl₃) δ 8.02 (dd, 1H), 7.58 (m, 2H), 7.48 (dd, 1H), 7.29 (m, 2H), 7.01 (dd, 1H), 6.82 (dd, 1H), 3.74 (s, 3H), 3.55 (br.s, 2H).

### Intermediate 31 3-[(5-chloro-3-nitro-2-pyridinyl)amino]benzoic acid

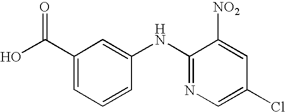

Intermediate 2 (500 mg, 2.59 mmol), 3-aminobenzoic acid (391 mg, 2.85 mmol) and triethylamine (1.1 mL, 7.77 mmol) were dissolved in 1,4-dioxane (10 mL) and heated to 100° C. for 18 h. The reaction mixture was dissolved in 30 mL of EtOAc (ethyl acetate) and water (30 mL) and acidified to <pH 3.0 with 2N HCl. The organic layer was separated, dried and concentrated in vacuo to afford the title compound as 710 mg (93% yield) of a yellow foam; ¹H NMR (d₆-DMSO) δ 13.03 (br s, 1H), 10.04 (s, 1H), 8.63 (d, 1H, 2.5 Hz), 8.59 (d, 1H, 2.3 Hz), 8.18 (t, 1H, 1.7 Hz), 7.82 (dd, 1H, 1.5, 7.2 Hz), 7.73 (d, 1H, 7.8 Hz), 7.50 (t, 7.8 Hz).

The following intermediates were prepared employing a similar method to that described for intermediate 31 above. Additional purification was needed for intermediate 32 using preparative HPLC (Phenomenex 75×30 mm column, 40 mL/min flow rate, A: 0.1% TFA in acetonitrile B: 0.1% TFA in water, A: 20 to 100% over 15 min, UV detection at 215 nm). As is appreciated by those skilled in the art, these analogous intermediates may involve routine variations in synthetic procedure.

| Inter- | | | | |
|---|---|---|---|---|
| mediate | | | | |
| No. | Precursor | Structure | Data | Name |
| 32 | 1H-1,2,4-triazol-3-amine | 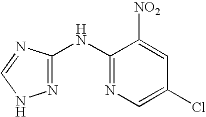 | MH⁺241/243 | 5-chloro-3-nitro-N-1H-1,2,4-triazol-3-yl-2-pyridinamine |

### Intermediate 33 5-chloro-2-[(2-methyl-3-pyridinyl)oxy]-3-pyridinamine

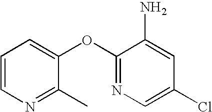

A solution of 5-chloro-2-[(2-methyl-3-pyridinyl)oxy]-3-nitropyridine (intermediate 6) (ca 2 mmol) in DCM (5 mL) was added to 5% platinum on carbon (Degussa-type, 312 mg), ammonium formate (1.5 g) was added and the mixture heated at reflux for 24 hours. The reaction mixture was filtered through Celite (2×2 mL, DCM wash) and the filtrate concentrated to give 390 mg of the product, apparently containing ca 10% of the intermediate hydroxylamine (characteristic peaks at 7.5-7.6 ppm); ¹H NMR (CDCl₃) 8.41 (dd, 1H), 7.43 (m, 2H), 7.21 (m, 2H), 7.04 (d, 1H), 4.22 (br.s, 2H), 2.45 (s, 3H). The reaction can be driven to completion by resubmitting the product to the above conditions.

The following intermediates were prepared using a method analogous to that described above for intermediate 33. In some cases H₂ gas at atmospheric pressure was used rather than ammonium formate.

| Intermediate | | | | |
|---|---|---|---|---|
| No. | From | Structure | Data | Name |
| 34 | Intermediate 7 | 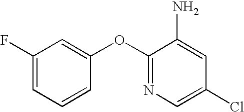 | MH⁺; 239 | 5-chloro-2-[(3-fluorophenyl)oxy]-3-pyridinamine |
| 35 | Intermediate 8 | 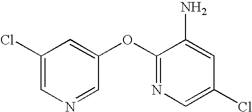 | MH⁺; 256 | 5-chloro-2-[(5-chloro-3-pyridinyl)oxy]-3-pyridinamine |
| 36 | Intermediate 9 | 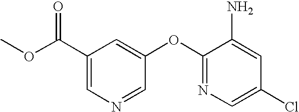 | MH⁺; 280 | Methyl-5-[(3-amino-5-chloro-2-pyridinyl)oxy]-3-pyridinecarboxylate |
| 37 | Intermediate 10 | 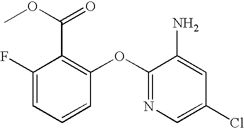 | MH⁺; 297 | methyl 2-[(3-amino-5-chloro-2-pyridinyl)oxy]-6-fluorobenzoate |
| 38 | Intermediate 11 | 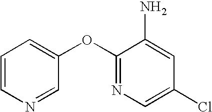 | MH⁺; 221 | 5-chloro-2-(3-pyridinyloxy)-3-pyridinamine |
| 39 | Intermediate 12 | 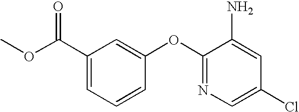 | MH⁺; 279 | methyl 3-[(3-amino-5-chloro-2-pyridinyl)oxy]benzoate |
| 40 | Intermediate 13 | 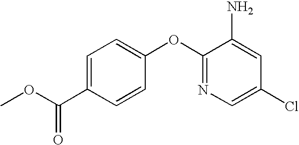 | MH⁺; 279 | methyl 4-[(3-amino-5-chloro-2-pyridinyl)oxy]benzoate |
| 41 | Intermediate 14 | 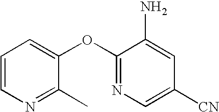 | MH⁺; 227 | 5-amino-6-[(2-methyl-3-pyridinyl)oxy]-3-pyridinecarbonitrile |
| 42 | Intermediate 15 | 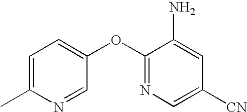 | MH⁺; 227 | 5-amino-6-[(6-methyl-3-pyridinyl)oxy]-3-pyridinecarbonitrile |
| 43 | Intermediate 16 | 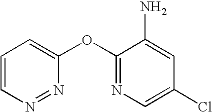 | MH⁺; 223 | 5-chloro-2-(3-pyridazinyloxy)-3-pyridinamine |
| 44 | Intermediate 17 | 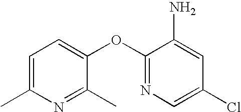 | MH⁺; 250 | 5-chloro-2-[(2,6-dimethyl-3-pyridinyl)oxy]-3-pyridinamine |
| 45 | 5-chloro-2-[(4-methyl-2-pyridinyl)oxy]-3-nitropyridine | 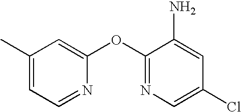 | MH⁺; 236 | 5-chloro-2-[(4-methyl-2-pyridinyl)oxy]-3-pyridinamine |
| 46 | Intermediate 18 | 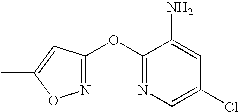 | MH⁺; 226 | 5-chloro-2-[(5-methyl-3-isoxazolyl)oxy]-3-pyridinamine |
| 47 | Intermediate 19 | 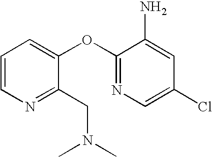 | MH⁺; 279 | 5-chloro-2-({2-[(dimethylamino)methyl]-3-pyridinyl}oxy)-3-pyridinamine |
| 48 | Intermediate 20 | 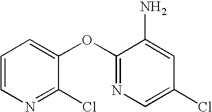 | MH⁺; 256 | 5-chloro-2-[(2-chloro-3-pyridinyl)oxy]-3-pyridinamine |
| 49 | Intermediate 21 | 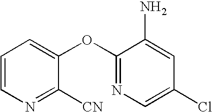 | MH⁺; 247 | 3-[(3-amino-5-chloro-2-pyridinyl)oxy]-2-pyridinecarbonitrile |
| 50 | Intermediate 22 | 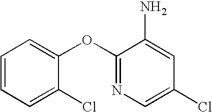 | MH⁺; 255 | {5-chloro-2-[(2-chlorophenyl)oxy]phenyl}amine |
| 51 | Intermediate 23 | 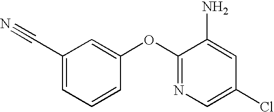 | MH⁺; 246 | 3-[(3-amino-5-chloro-2-pyridinyl)oxy]benzonitrile |
| 52 | Intermediate 24 | 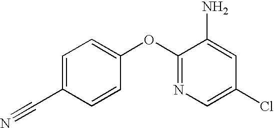 | MH⁺; 246 | 4-[(3-amino-5-chloro-2-pyridinyl)oxy]benzonitrile |
| 53 | Intermediate 25 | 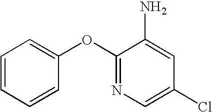 | MH⁺; 222 | 5-chloro-2-(phenyloxy)-3-pyridinamine |
| 54 | 4-{[(5-chloro-3-nitro-2-pyridinyl)oxy]acetyl}morpholine | 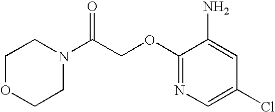 | MH⁺; 272 | 5-chloro-2-{[2-(4-morpholinyl)-2-oxoethyl]oxy}-3-pyridinamine |
| 55 | 2-[(5-chloro-3-nitro-2-pyridinyl)oxy]-N-[2-(dimethylamino)ethyl]-N-methylacetamide | 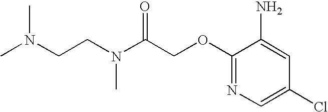 | MH⁺; 287 | 2-[(3-amino-5-chloro-2-pyridinyl)oxy]-N-[2-(dimethylamino)ethyl]-N-methylacetamide |
| 56 | 5-chloro-2-[(1-methyl-1H-tetrazol-5-yl)oxy]-3-nitropyridine | 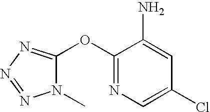 | MH⁺; 227 | 5-chloro-2-[(1-methyl-1H-tetrazol-5-yl)oxy]-3-pyridinamine |
| 57 | Intermediate 31 | 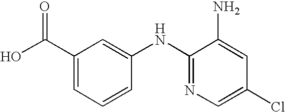 | MH⁺; 264 | 3-[(3-amino-5-chloro-2-pyridinyl)amino]benzoic acid |
| 58 | Intermediate 32 | 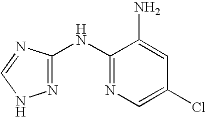 | MH⁺211/213 | 5-chloro-N²-1H-1,2,4-triazol-3-yl-2,3-pyridinediamine |

### Intermediate 59 5-chloro-2-{[2-(4-morpholinylmethyl)-3-pyridinyl]oxy}-3-pyridineamine

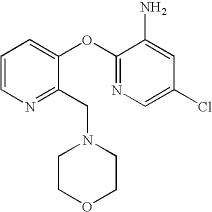

A solution of 2-(4-morpholinylmethyl)-3-pyridinol (251 mg) in DMF (2.5 mL) was prepared and added to 2,5-dichloro-3-nitropyridine (238 mg). Potassium carbonate (255 eq) was added and the mixture heated to 90° C. for 2 hours. The mixture was then cooled and concentrated. The resulting solid was partitioned between DCM and water (5 mL each) and separated. The DCM layer was concentrated and re-dissolved in DCM (3.3 mL) then added to 5% platinum on carbon (Degussa-type, 203 mg). Ammonium formate (1.0 g) was added and the mixture heated at reflux for 24 hours. The reaction mixture was filtered through Celite (2×2 mL DCM wash) and the filtrate concentrated to give 224 mg of the product; ¹H NMR (CDCl₃) 8.45 (d, 1H), 7.51 (m, 2H), 7.26 (m, 2H), 7.02 (d, 1H), 4.58 (br.s, 2H), 3.76 (s, 2H), 3.53 (m, 4H), 2.46 (m, 4H).

The following aniline intermediates were prepared employing a method analogous to that described above for intermediate 59.

| Intermediate | | | | |
|---|---|---|---|---|
| No. | Precursor(s) | Structure | Data | Name |
| 60 | 5-hydroxy-2-pyridinecarbonitrile& 2,5-dichloro-3-nitropyridine | 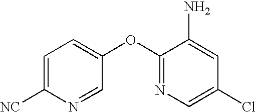 | MH⁺; 248 | 5-[(3-amino-5-chloro-2-pyridinyl)oxy]-2-pyridinecarbonitrile |
| 61 | methyl 3-hydroxy-2-pyridinecarboxylate& 2,5-dichloro-3-nitropyridine | 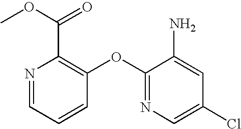 | MH⁺; 281 | methyl 3-[(3-amino-5-chloro-2-pyridinyl)oxy]-2-pyridinecarboxylate |
| 62 | 6-methyl-3-hydroxypyridine& 2,5-dichloro-3-nitropyridine | 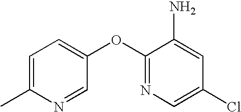 | MH⁺; 237 | 5-chloro-2-[(6-methyl-3-pyridinyl)oxy]-3-pyridinamine |

### Intermediate 63 methyl 5-[(5-chloro-3-{[(3,4-dichlorophenyl)sulfonyl]amino}-2-pyridinyl)oxy]-3-pyridinecarboxylate

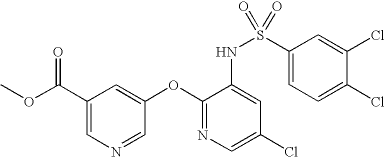

A mixture of intermediate 22 (5 mg) and 3,4-dichlorobenzenesulfonyl chloride (109 uL) in pyridine (2 mL) was heated to 95° C. for 1 hour, cooled and concentrated. The product was dissolved in methanol and retained on an aminopropyl SPE cartridge (5 g). The cartridge was washed with methanol (10 mL) and the product eluted with 2M ammonia in methanol. The eluent was concentrated and partitioned between ethyl acetate and water and the organic layer concentrated to provide 180 mg of the product; [M]⁻=488; ¹H NMR (CDCl₃) δ 9.12 (d, 1H), 8.45 (d, 1H), 7.96 (m, 2H), 7.85 (m, 2H), 7.65 (dd, 1H), 7.56 (d, 1H), 3.97 (s, 3H).

The intermediates listed in the table below were prepared employing a method analogous to that described above for intermediate 64 using appropriate staring materials.

| Intermediate | | | | |
|---|---|---|---|---|
| No. | From | Structure | Data | Name |
| 64 | Intermediate 34 | 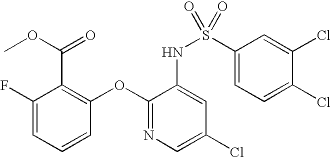 | MH⁺; 505 | methyl2-[(5-chloro-3-{[(3,4-dichlorophenyl)sulfonyl]amino}-2-pyridinyl)oxy]-6-fluorobenzoate |
| 65 | Intermediate 39 | 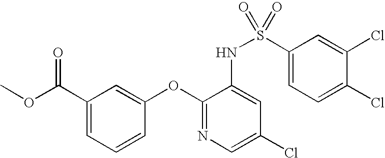 | MH⁺; 488 | methyl 3-[(5-chloro-3-{[(3,4-dichlorophenyl)sulfonyl]amino}-2-pyridinyl)oxy]-benzoate |
| 66 | Intermediate 39 | 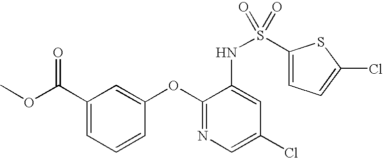 | MH⁺; 458 | methyl 3-[(5-chloro-3-{[(5-chloro-2-thienyl)sulfonyl]amino}-2-pyridinyl)oxy]-benzoate |
| 67 | Intermediate 40 | 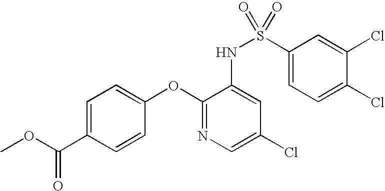 | MH⁺; 488 | methyl 4-[(5-chloro-3-{[(3,4-dichlorophenyl)sulfonyl]amino}-2-pyridinyl)oxy]-benzoate |
| 68 | Intermediate 40 | 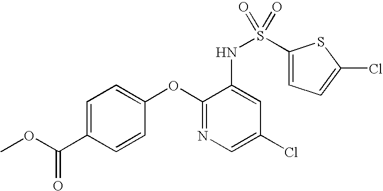 | MH⁺; 458 | methyl 4-[(5-chloro-3-{[5-chloro-2-thienyl)sulfonyl]amino}-2-pyridinyl)oxy]-benzoate |
| 69 | Intermediate 61 | 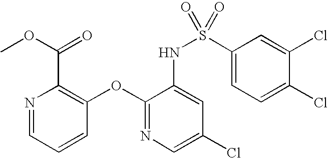 | MH⁺; 490 | methyl 3-[(5-chloro-3-{[(3,4-dichlorophenyl)sulfonyl]amino}-2-pyridinyl)oxy]-2-pyridine-carboxylate |
| 70 | Intermediate 61 | 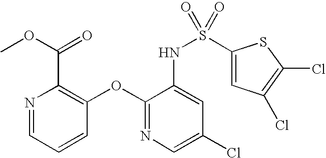 | MH⁺; 490 | methyl 3-[(5-chloro-3-{[(3,4-dichlorophenyl)sulfonyl]amino}-2-pyridinyl)oxy]-2-pyridine-carboxylate |
| 71 | Intermediate 29 | 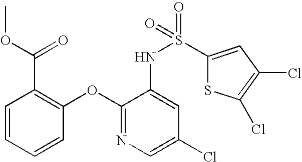 | MH⁺; 495 | methyl 2-[(5-chloro-3-{[(4,5-dichloro-2-thienyl)sulfonyl]amino}-2-pyridinyl)oxy]-benzoate |
| 72 | Intermediate 29 | 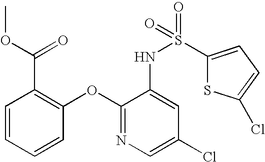 | MH⁺; 459 | methyl 2-[(5-chloro-3-{[(5-chloro-2-thienyl)sulfonyl]amino}-2-pyridinyl)oxy]benzoate |
| 73 | Intermediate 29 | 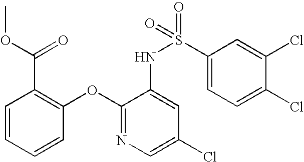 | MH⁺; 489 | methyl 2-[(5-chloro-3-{[3,4-dichlorophenyl)sulfonyl]amino}-2-pyridinyl)oxy]-benzoate |

### Intermediate 74 2-chloropyrido[2,3-b][1,4]benzoxazepin-6(5H)-one

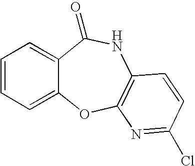

To a stirred solution of intermediate 29 (917 mg) in 20 mL of anhydrous THF (tetrahydrofuran) was added potassium tert-butoxide (443 mg) in one portion. The resultant purple solution was heated at 60° C. for 2 hours, cooled and partitioned between chloroform and water (20 ml each). The layers were separated and the aqueous layer extracted with further chloroform (150 ml in total), the combined organic layers were washed with water, eluted through an isolute phase separator and concentrated in vacuo to afford the title compound; 560 mg (69%); MH+ 247.

### Intermediate 75 2-chloro-5-[(3,4-dichlorophenyl)sulfonyl]pyrido[2,3-b][1,4]benzoxazepin-6(5H)-one

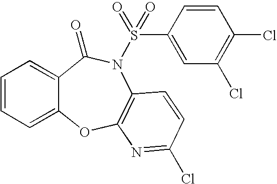

To a suspension of sodium hydride (60% mineral oil dispersion, 13 mg) in anhydrous THF (15 ml) at 0° C. was added intermediate 74 (350 mg) portion-wise. The resultant purple solution was stirred at 0° C. for 15 minutes and then 3,4-dichlorobenzenesulfonyl chloride (265 ul) was added in one portion. The mixture was stirred for a further 30 minutes at room temperature and then 2 M hydrochloric acid (2 ml) was added. The crude reaction mixture was concentrated in vacuo to give a yellow solid which was suspended in ethyl acetate and chloroform the inorganic salts were filtered off and the liquors were concentrated in vacuo to afford 1.03 g of the title compound; MH+=457.

### EXAMPLES

### Example 1

### 2-[(5-chloro-3-{[(3,4-dichlorophenyl)sulfonyl]amino}-2-pyridinyl)oxy]benzoic acid

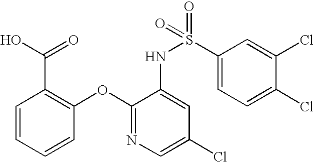

To a suspension of intermediate 73 (115 mg) in water (5 ml) and methanol (5 ml) was added lithium hydroxide (23 mg). The resultant solution was stirred at 30° C. for 2 hours. The crude reaction mixture was concentrated in vacuo to yield a white solid which was partitioned between water and diethyl ether (10 ml each). The layers were separated and the basic aqueous layer was acidified to pH 1 using 2N Hydrochloric acid and then extracted with chloroform (3×20 ml). The combined organics were washed with 1N Hydrochloric acid, water, dried and concentrated in vacuo to afford the title compound, 107 mg (96%), MH+ 475.

### Example 2

### 5-[(5-chloro-3-{[(3,4-dichlorophenyl)sulfonyl]amino}-2-pyridinyl)oxy]-3-pyridinecarboxylic acid

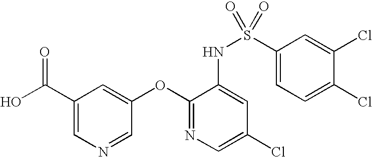

To a suspension of intermediate 63 (180 mg) in methanol (1.5 mL) was added 4M lithium hydroxide solution (3.7 mL) and the mixture warmed to 50° C. for 15 minutes and then allowed to cool to room temperature overnight. The mixture volume was reduced by 50% under a stream of nitrogen, acidified (2M HCl; to pH 2) and extracted into ethyl acetate, dried (saturated brine and sodium sulfate) and blown down to give the product (125 mg).

[M]⁻=476.

The compounds in the table below were similarly prepared using a method analogous to that described above for example 2. In some instances it proved necessary to repeat the reaction with further equivalents of lithium hydroxide or alternatively to allow a longer reaction time at 50° C.

| Eg | Structure | From | +ve | −ve | Name |
|---|---|---|---|---|---|
| 3 | 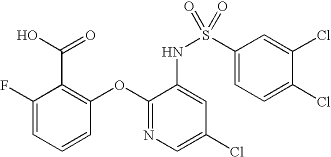 | Interm. 64 | 493 | 491 | 2-(5-chloro-3-{[(3,4-dichlorophenyl)sulfonyl]amino}-2-pyridinyl)oxy]-6-fluorobenzoic acid |
| 4 | 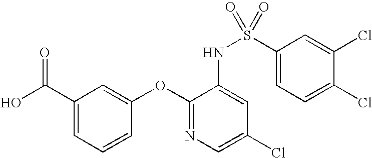 | Interm. 65 | − | 472 | 3-[(5-chloro-3-{[(3,4-dichlorophenyl)sulfonyl]amino}-2-pyridinyl)oxy]benzoicacid |
| 5 | 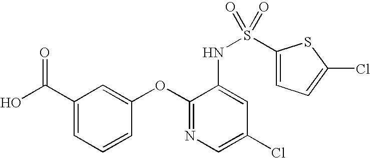 | Interm. 66 | − | 443 | 3-[(5-chloro-3-{[(5-chloro-2-thienyl)sulfonyl]amino}-2-pyridinyl)oxy]benzoicacid |
| 6 | 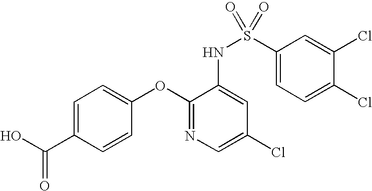 | Interm. 67 | − | 472 | 4-[(5-chloro-3-{[(3,4-dichlorophenyl)sulfonyl]amino}-2-pyridinyl)oxy]benzoicacid |
| 7 | 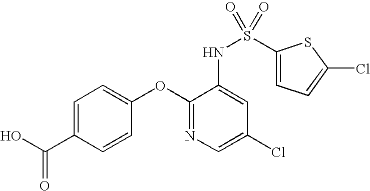 | Interm. 68 | − | 443 | 4-[(5-chloro-3-{[(5-chloro-2-thienyl)suIfonyl]amino}-2-pyridinyl)oxy]benzoicacid |
| 8 | 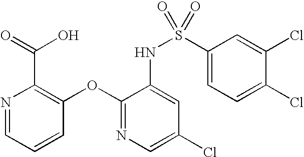 | Interm. 69 | − | 474 | 3-[(5-chloro-3-{[(3,4-dichlorophenyl)sulfonyl]amino}-2-pyridinyl)oxy]-2-pyridinecarboxylicacid |
| 9 | 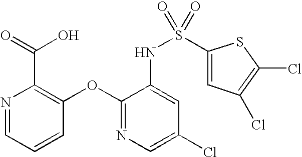 | Interm. 70 | − | 480 | 3-[(5-chloro-3-{[(4,5-dichloro-2-thienyl)sulfonyl]amino}-2-pyridinyl)oxy]-2-pyridinecarboxylic acid |
| 10 | 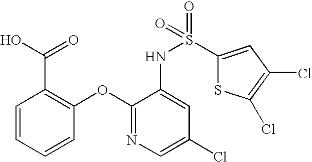 | Interm. 71 | 481 | 479 | 2-[(5-chloro-3-{[(4,5-dichloro-2-thienyl)sulfonyl]amino}-2-pyridinyl)oxy]benzoicacid |
| 11 | 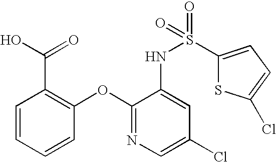 | Interm. 72 | 445 | 443 | 2-[(5-chloro-3-{[(5-chloro-2-thienyl)sulfonyl]amino}-2-pyridinyl)oxy]benzoicacid |

### Example 12

### 3,4-dichloro-N-(5-chloro-2-{[4-(hydroxymethyl)phenyl]oxy}-3-pyridinyl)benzenesulfonamide

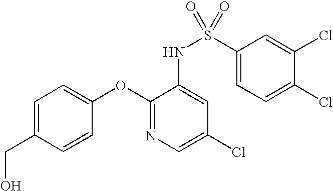

A solution of dry THF (2.1 mL) was added to a dry two-necked flask with Example 6 (600 mg). The flask was cooled down to 0° C. and then a 1.0 M solution of lithium aluminum hydride (2.53 mL) in THF? was added dropwise. The reaction was brought to room temperature and then stirred for one hour. Once the reaction was complete it was brought back to 0° C. and quenched. The precipitate was filtered off and then the precipitant was concentrated and purified on an ISCO MPLC to yield 202 mg of the title compound, MH+; 460.

The following intermediate was made using a method analogous to that for example 12.

| Example | Structure | From | +ve | −ve | Name |
|---|---|---|---|---|---|
| 13 | 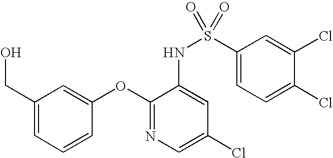 | Example 4 | 461 | | 3,4-dichloro-N-(5-chloro-2-{[3-(hydroxymethyl)phenyl]oxy}-3-pyridinyl)benzene-sulfonamide |

### Intermediate 76 3,4-dichloro-N-{5-chloro-2-[(4-formylphenyl)oxy]-3-pyridinyl}benzenesulfonamide

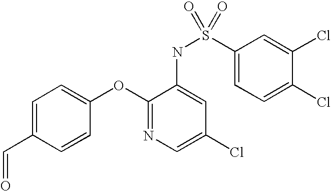

Example 12 (202 mg) was dissolved in dry DCM (5 mL) and then resin bound MP-TsO-TEMPO (loading=1 mmol/1 g, 878 mg) was added and stirred at room temperature overnight. When the reaction was done the resin was filtered off and the precipitant was concentrated. ISCO MPLC was used if needed for further purification to yield 216 mg, MH+; 578.

The following intermediate was made using a method analogous to that for intermediate 76.

| Intermediate | Structure | From | +ve | −ve | Name |
|---|---|---|---|---|---|
| 77 | 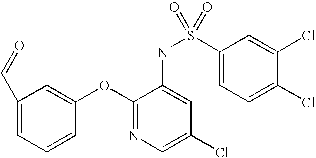 | Example 13 | 459 | − | 3,4-dichloro-N-{5-chloro-2-[(3-formylphenyl)oxy]-3-pyridinyi}benzene-sulfonamide |

### Example 14

### 3,4-dichloro-N-{5-chloro-2-[(2-methyl-3-pyridinyl)oxy]-3-pyridinyl}benzenesulfonamide

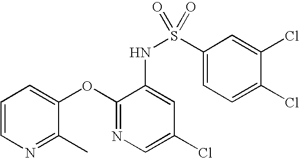

A mixture of intermediate 33 (165 mg), 4-(dimethylamino)-pyridine (5 mg) and 3,4-dichlorobenzenesulfonyl chloride (109 uL) in pyridine (2 mL) was heated to 95° C. for 1 hour, cooled and blown down. The product was dissolved in methanol and retained on an aminopropyl SPE cartridge (5 g). The cartridge was washed with methanol (10 mL) and the product eluted with 2M ammonia in methanol. The eluent was blown down and partitioned between chloroform (ethyl acetate may also be used) and water and the organic layer blown down to provide 155 mg of the product. [MH]⁺=444

The following examples were prepared using appropriate starting materials by a method analogous to that described above for Example 14 followed by purification using silica-SPE with appropriate solvents and/or mass-directed auto-preparative HPLC. In some cases, a reaction took place at a lower temperature. Variations in reaction time also occurred.

| Eg | Structure | From | +ve | −ve | Name |
|---|---|---|---|---|---|
| 15 | 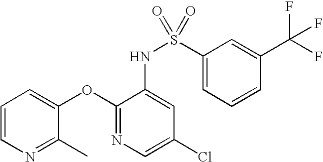 | Intermediate 33 | 444 | 442 | N-{5-chloro-2-[(2-methyl-3-pyridinyl)oxy]-3-pyridinyl}-3-(trifluoromethyl)benzene-sulfonamide |
| 16 | 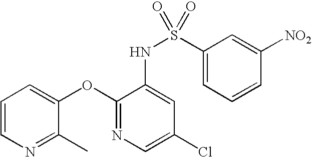 | Intermediate 33 | 421 | | N-{5-chloro-2-[(2-methyl-3-pyridinyl)oxy]-3-pyridinyl}-3-nitrobenzenesulfonamide |
| 17 | 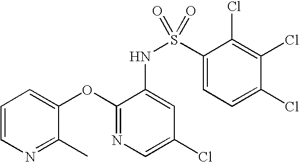 | Intermediate 33 | 478 | | 2,3,4-trichloro-N-{5-chloro-2-[(2-methyl-3-pyridinyl)oxy]-3-pyridinyl}benzenesulfonamide |
| 18 | 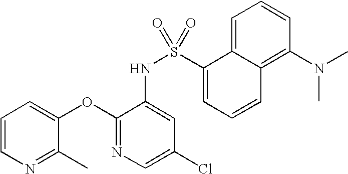 | Intermediate 33 | 469 | | N-{5-chloro-2-[(2-methyl-3-pyridinyl)oxy]-3-pyridinyl}-5-(dimethylamino)-1-naphthalenesulfonamide |
| 19 | 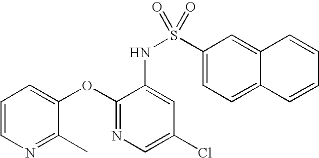 | Intermediate 33 | 426 | | N-{5-chloro-2-[(2-methyl-3-pyridinyl)oxy]-3-pyridinyl}-2-naphthalenesulfonamide |
| 20 | 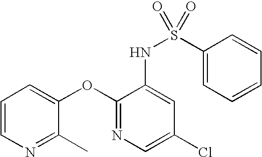 | Intermediate 33 | 376 | | N-{5-chloro-2-[(2-methyl-3-pyridinyl)oxy]-3-pyridinyl}benzenesulfonamide |
| 21 | 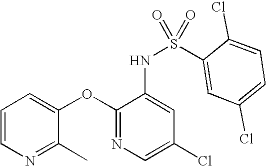 | Intermediate 33 | 444 | | 2,5-dichloro-N-{5-chloro-2-[(2-methyl-3-pyridinyl)oxy]-3-pyridinyl}benzenesulfonamide |
| 22 | 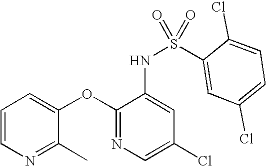 | Intermediate 33 | 460 | | 3,5-dichloro-N-{5-chloro-2-[(2-methyl-3-pyridinyl)oxy]-3-pyridinyl}-2-hydroxybenzenesulfonamide |
| 23 | 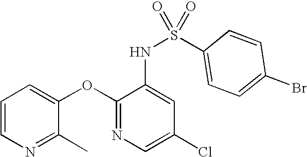 | Intermediate 33 | 455 | | 4-bromo-N-{5-chloro-2-[(2-methyl-3-pyridinyl)oxy]-3-pyridinyl}benzenesulfonamide |
| 24 | 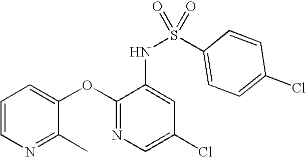 | Intermediate 33 | 410 | | 4-chloro-N-{5-chloro-2-[(2-methyl-3-pyridinyl)oxy]-3-pyridinyl}benzenesulfonamide |
| 25 | 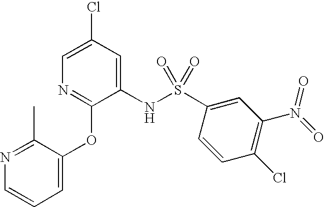 | Intermediate 33 | 455 | | 4-chloro-N-{5-chloro-2-[(2-methyl-3-pyridinyl)oxy]-3-pyridinyl}-3-nitrobenzenesulfonamide |
| 26 | 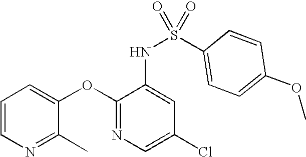 | Intermediate 33 | 406 | | N-{5-chloro-2-[(2-methyl-3-pyridinyl)oxy]-3-pyridinyl}-4-(methyloxy)benzene-sulfonamide |
| 27 | 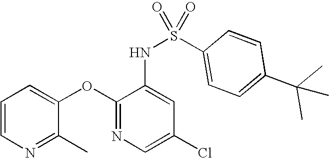 | Intermediate 33 | 432 | | N-{5-chloro-2-[(2-methyl-3-pyridinyl)oxy]-3-pyridinyl}-4-(1,1-dimethyl-ethyl)benzenesulfonamide |
| 28 | 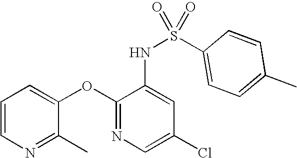 | Intermediate 33 | 390 | | N-{5-chloro-2-[(2-methyl-3-pyridinyl)oxy]-3-pyridinyl}-4-methylbenzenesulfonamide |
| 29 | 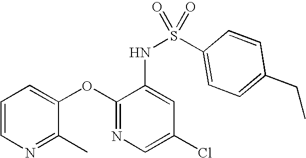 | Intermediate 33 | 404 | | N-{5-chloro-2-[(2-methyl-3-pyridinyl)oxy]-3-pyridinyl}-4-ethylbenzenesulfonamide |
| 30 | 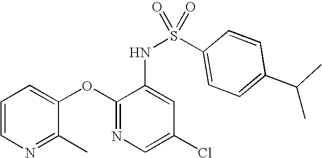 | Intermediate 33 | 418 | | N-{5-chloro-2-[(2-methyl-3-pyridinyl)oxy]-3-pyridinyl}-4-(1-methylethyl)benzenesulfonamide |
| 31 | 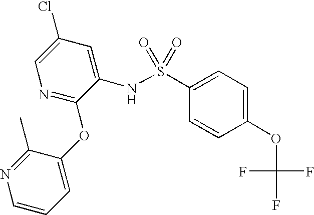 | Intermediate 33 | 460 | | N-{5-chloro-2-[(2-methyl-3-pyridinyl)oxy]-3-pyridinyl}-4-[(trifluoromethyl)oxy]benzene-sulfonamide |
| 32 | 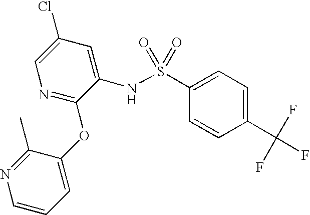 | Intermediate 33 | 444 | | N-{5-chloro-2-[(2-methyl-3-pyridinyl)oxy]-3-pyridinyl}-4-(trifluoromethyl)benzenesul-fonamide |
| 33 | 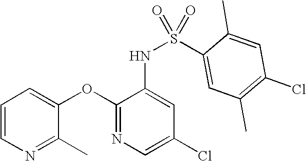 | Intermediate 33 | 438 | | 4-chloro-N-{5-chloro-2-[(2-methyl-3-pyridinyl)oxy]-3-pyridinyl}-2,5-dimethylbenzenesulfonamide |
| 34 | 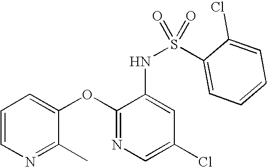 | Intermediate 33 | 410 | | 2-chloro-N-{5-chloro-2-[(2-methyl-3-pyridinyl)oxy]-3-pyridinyl}benzenesulfonamide |
| 35 | 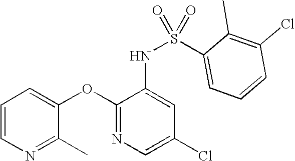 | Intermediate 33 | 424 | | 3-chloro-N-{5-chloro-2-[(2-methyl-3-pyridinyl)oxy]-3-pyridinyl}-2-methylbenzenesulfonamide |
| 36 | 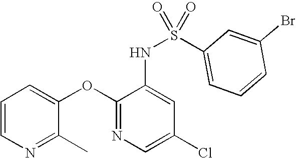 | Intermediate 33 | 455 | | 3-bromo-N-{5-chloro-2-[(2-methyl-3-pyridinyl)oxy]-3-pyridinyl}benzenesulfonamide |
| 37 | 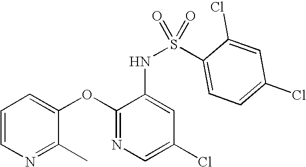 | Intermediate 33 | 444 | | 2,4-dichloro-N-{5-chloro-2-[(2-methyl-3-pyridinyl)oxy]-3-pyridinyl}benzenesulfonamide |
| 38 | 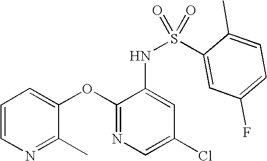 | Intermediate 33 | 408 | | N-{5-chloro-2-[(2-methyl-3-pyridinyl)oxy]-3-pyridinyl}-5-fluoro-2-methylbenzenesulfonamide |
| 39 | 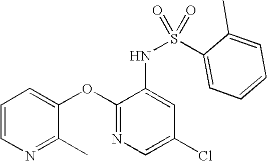 | Intermediate 33 | 390 | | N-{5-chloro-2-[(2-methyl-3-pyridinyl)oxy]-3-pyridinyl}-2-methylbenzenesulfonamide |
| 40 | 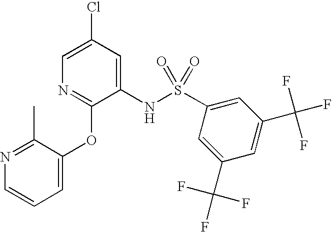 | Intermediate 33 | 512 | | N-{5-chloro-2-[(2-methyl-3-pyridinyl)oxy]-3-pyridinyl}-3,5-bis(trifluoromethyl)benzene-sulfonamide |
| 41 | 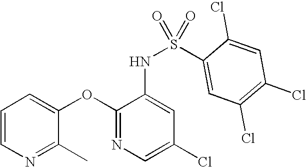 | Intermediate 33 | 478 | | 2,4,5-trichloro-N-{5-chloro-2-[(2-methyl-3-pyridinyl)oxy]-3-pyridinyl}benzenesulfonamide |
| 42 | 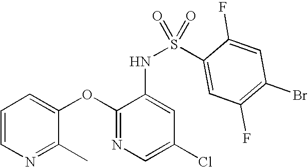 | Intermediate 33 | 491 | | 4-bromo-N-{5-chloro-2-[(2-methyl-3-pyridinyl)oxy]-3-pyridinyl}-2,5-difluorobenzenesulfonamide |
| 43 | 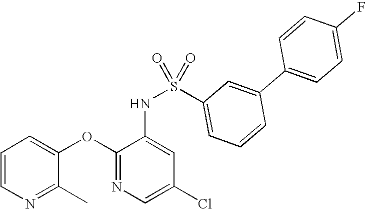 | Intermediate 33 | 470 | | N-{5-chloro-2-[(2-methyl-3-pyridinyl)oxy]-3-pyridinyl}-4′-fluoro-3-biphenylsulfonamide |
| 44 | 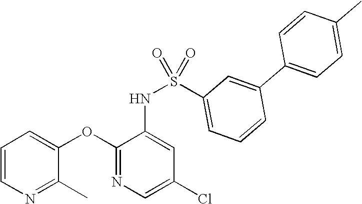 | Intermediate 33 | 466 | | N-{5-chloro-2-[(2-methyl-3-pyridinyl)oxy]-3-pyridinyl}-4′-methyl-3-biphenylsulfonamide |
| 45 | 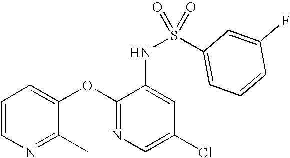 | Intermediate 33 | 394 | | N-{5-chloro-2-[(2-methyl-3-pyridinyl)oxy]-3-pyridinyl}-3-fluorobenzenesulfonamide |
| 46 | 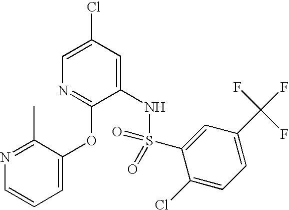 | Intermediate 33 | 478 | | 2-chloro-N-{5-chloro-2-[(2-methyl-3-pyridinyl)oxy]-3-pyridinyl}-5-(trifluoromethyl)benzene-sulfonamide |
| 47 | 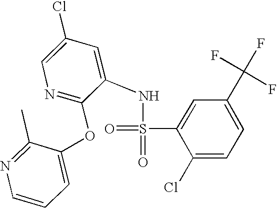 | Intermediate 33 | 478 | | 2-chloro-N-{5-chloro-2-[(2-methyl-3-pyridinyl)oxy]-3-pyridinyl}-4-(trifluoromethyl)benzene-sulfonamide |
| 48 | 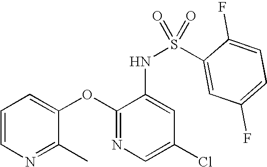 | Intermediate 33 | 412 | | N-{5-chloro-2-[(2-methyl-3-pyridinyl)oxy]-3-pyridinyl}-2,5-difluorobenzenesulfonamide |
| 49 | 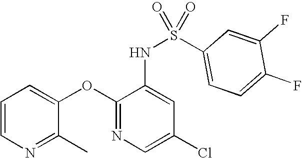 | Intermediate 33 | 412 | | N-{5-chloro-2-[(2-methyl-3-pyridinyl)oxy]-3-pyridinyl}-3,4-difluorobenzenesulfonamide |
| 50 | 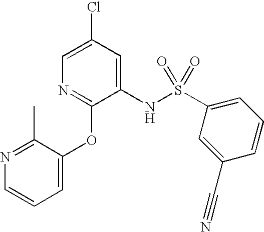 | Intermediate 33 | 401 | | N-{5-chloro-2-[(2-methyl-3-pyridinyl)oxy]-3-pyridinyl}-3-cyanobenzenesulfonamide |
| 51 | 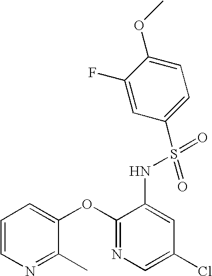 | Intermediate 33 | 424 | | N-{5-chloro-2-[(2-methyl-3-pyridinyl)oxy]-3-pyridinyl}-3-fluoro-4-(methyloxy)benzene-sulfonamide |
| 52 | 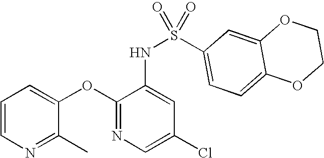 | Intermediate 33 | 434 | | N-{5-chloro-2-[(2-methyl-3-pyridinyl)oxy]-3-pyridinyl}-2,3-dihydro-1,4-benzodioxin-6-sulfonamide |
| 53 | 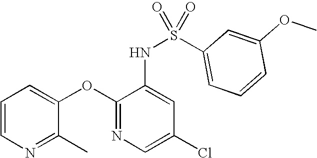 | Intermediate 33 | 406 | | N-{5-chloro-2-[(2-methyl-3-pyridinyl)oxy]-3-pyridinyl}-3-(methyloxy)benzenesulfonamide |
| 54 | 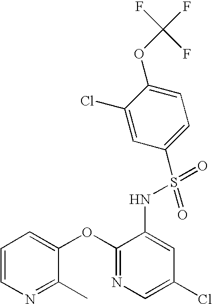 | Intermediate 33 | 494 | | 3-chloro-N-{5-chloro-2-[(2-methyl-3-pyridinyl)oxy]-3-pyridinyl}-4-[(trifluoromethyl)oxy]benzene-sulfonamide |
| 55 | 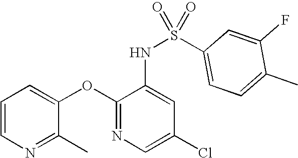 | Intermediate 33 | 408 | | N-{5-chloro-2-[(2-methyl-3-pyridinyl)oxy]-3-pyridinyl}-3-fluoro-4-methylbenzenesulfonamide |
| 56 | 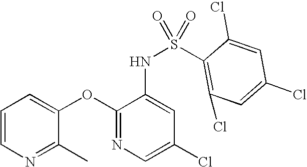 | Intermediate 33 | 478 | | 2,4,6-trichloro-N-{5-chloro-2-[(2-methyl-3-pyridinyl)oxy]-3-pyridinyl}benzenesulfonamide |
| 57 | 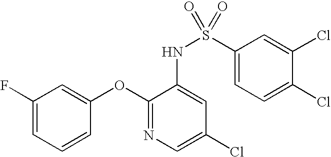 | Intermediate 34 | 449 | − | 3,4-dichloro-N-{5-chloro-2-[(3-fluorophenyl)oxy]-3-pyridinyl}benzenesulfonamide |
| 58 | 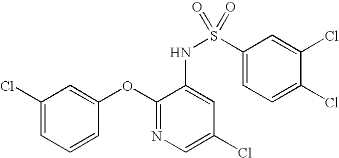 | Intermediate 35 | 466 | 464 | 3,4-dichloro-N-{5-chloro-2-[(5-chloro-3-pyridinyl)oxy]-3-pyridinyl}benzenesulfonamide |
| 59 | 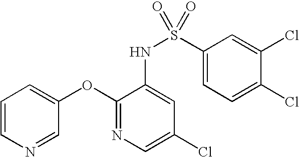 | Intermediate 38 | 431 | − | 3,4-dichloro-N-[5-chloro-2-(3-pyridinyloxy)-3-pyridinyl]benzenesuifonamide |
| 60 | 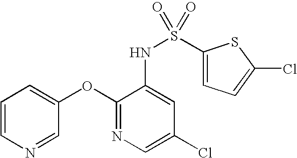 | Intermediate 38 | − | 401 | 5-chloro-N-[5-chloro-2-(3-pyridinyloxy)-3-pyridinyl]-2-thiophenesulfonamide |
| 61 | 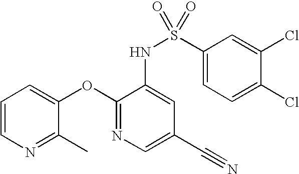 | Intermediate 41 | 435 | − | 3,4-dichloro-N-{5-cyano-2-[(2-methyl-3-pyridinyl)oxy]-3-pyridinyl}benzenesulfonamide |
| 62 | 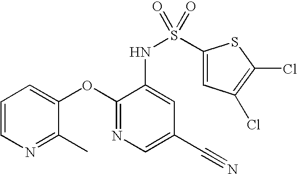 | Intermediate 41 | 441 | − | 4,5-dichloro-N-{5-cyano-2-[(2-methyl-3-pyridinyl)oxy]-3-pyridinyl}-2-thiophenesulfonamide |
| 63 | 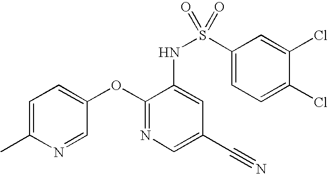 | Intermediate 42 | 435 | − | 3,4-dichloro-N-{5-cyano-2-[(6-methyl-3-pyridinyl)oxy]-3-pyridinyl}benzenesulfonamide |
| 64 | 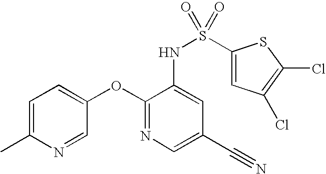 | Intermediate 42 | 441 | − | 4,5-dichloro-N-{5-cyano-2-[(6-methyl-3-pyridinyl)oxy]-3-pyridinyl}-2-thiophenesulfonamide |
| 65 | 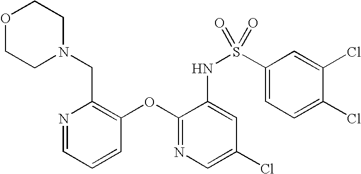 | Intermediate 59 | 530 | − | 3,4-dichloro-N-(5-chloro-2-{[2-(4-morpholinylmethyl)-3-pyridinyl]oxy}-3-pyridinyl)benzenesulfonamide |
| 66 | 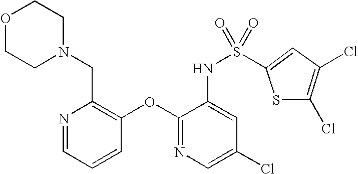 | Intermediate 59 | 537 | − | 4,5-dichloro-N-(5-chloro-2-{[2-(4-morpholinylmethyl)-3-pyridinyl]oxy}-3-pyridinyl)-2-thiophenesulfonamide |
| 67 | 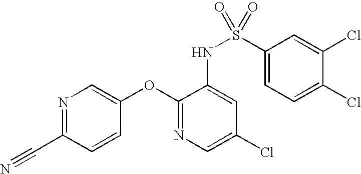 | Intermediate 60 | 457 | − | 3,4-dichloro-N-{5-chloro-2-[(6-cyano-3-pyridinyl)oxy]-3-pyridinyl}benzenesulfonamide |
| 68 | 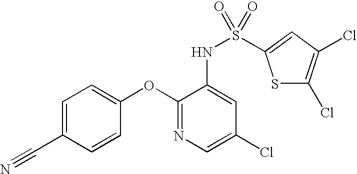 | Intermediate 60 | 463 | − | 4,5-dichloro-N-{5-chloro-2-[(6-cyano-3-pyridinyl)oxy]-3-pyridinyl}-2-thiophenesulfonamide |
| 69 | 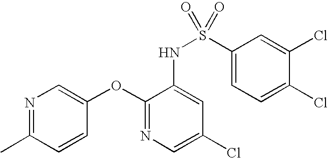 | Intermediate 62 | 446 | − | 3,4-dichloro-N-{5-chloro-2-{(6-methyl-3-pyridinyl)oxy]-3-pyridinyl}benzenesulfonmide |
| 70 | 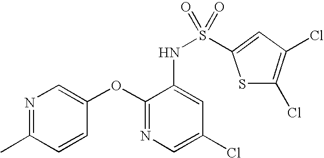 | Intermediate 62 | 452 | − | 4,5-dichloro-N-{5-chloro-2-[(6-methyl-3-pyridinyl)oxy]-3-pyridinyl}-2-thiophenesulfonamide |
| 71 | 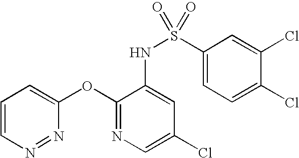 | Intermediate 43 | 433 | 431 | 3,4-dichloro-N-[5-chloro-2-(3-pyridazinyloxy)-3-pyridinyl]benzenesulfonamide |
| 72 | 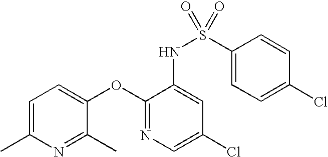 | Intermediate 44 | 424 | − | 4-chloro-N-{5-chloro-2-[(2,6-dimethyl-3-pyridinyl)oxy]-3-pyridinyl}benzenesulfonamide |
| 73 | 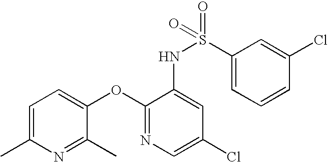 | Intermediate 44 | 424 | − | 3-chloro-N-{5-chloro-2-[(2,6-dimethyl-3-pyridinyl)oxy)-3-pyridinyl}benzenesulfonamide |
| 74 | 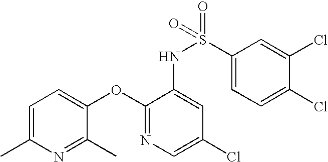 | Intermediate 44 | 458 | − | 3,4-dichloro-N-{5-chloro-2-[(2,6-dimethyl-3-pyridinyl)oxy]-3-pyridinyl}-benzenesulfonamide |
| 75 | 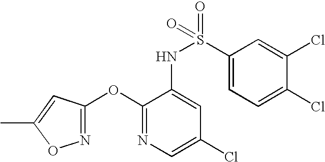 | Intermediate 46 | 434 | − | 3,4-dichloro-N-{5-chloro-2-[(5-methyl-3-isoxazolyl)oxy]-3-pyridinyl}benzenesulfonamide |
| 76 | 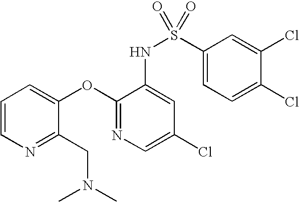 | Intermediate 47 | 489 | | 3,4-dichloro-N-[5-chloro-2-({2-[(dimethylamino)methyl]-3-pyridinyl}oxy)-3-pyridinyl]benzenesulfonamide |
| 77 | 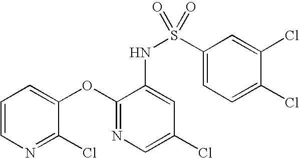 | Intermediate 48 | 466 | | 3,4-dichloro-N-{5-chloro-2-[(2-chloro-3-pyridinyl)oxy]-3-pyridinyl}benzenesulfonamide |
| 78 | 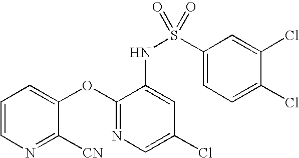 | Intermediate 49 | 457 | | 3,4-dichloro-N-{5-chloro-2-[(2-cyano-3-pyridinyl)oxy]-3-pyridinyl}benzenesulfonamde |
| 79 | 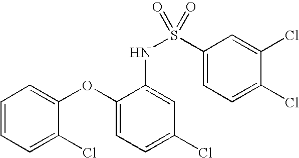 | Intermediate 50 | | 463 | 3,4-dichloro-N-{5-chloro-2-[(2-chlorophenyl)oxy]phenyl}-benzenesulfonamide |
| 80 | 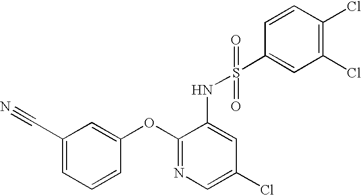 | Intermediate 51 | 455 | | 3,4-dichloro-N-{5-chloro-2-[(3-cyanophenyl)oxy]-3-pyridinyl}benzenesulfonamide |
| 81 | 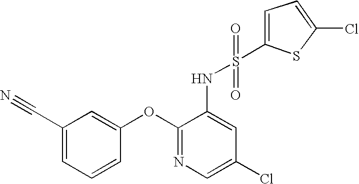 | Intermediate 51 | 426 | | 5-chloro-N-{5-chloro-2-[(3-cyanophenyl)oxy]-3-pyridinyl}-2-thiophenesulfonamide |
| 82 | 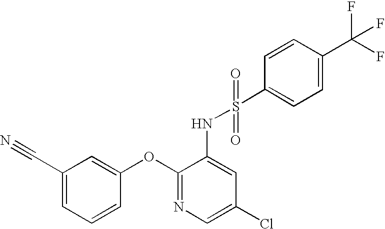 | Intermediate 51 | 454 | | N-{5-chloro-2-[(3-cyanophenyl)oxy]-3-pyridinyl}-4-(trifluoromethyl)benzene-sulfonamide |
| 83 | 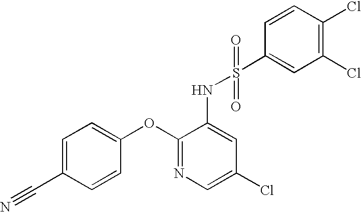 | Intermediate 52 | 454 | | 3,4-dichloro-N-{5-chloro-2-[(4-cyanophenyl)oxy]-3-pyridinyl}benzenesulfonamide |
| 84 | 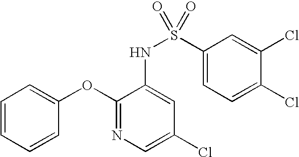 | Intermediate 53 | 431 | | 3,4-dichloro-N-[5-chloro-2-(phenyloxy)-3-pyridinyl]benzenesulfonamide |
| 85 | 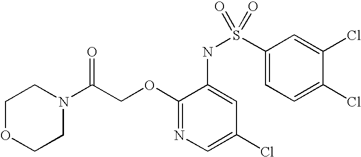 | Intermediate 54 | 481 | | 3,4-dichloro-N-(5-chloro-2-{[2-(4-morpholinyl)-2-oxoethyl]oxy}-3-pyridinyl)benzenesulfonamide |
| 86 | 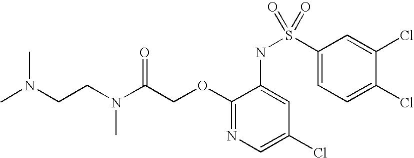 | Intermediate 55 | 496 | | 2-[(5-chloro-3-{[(3,4-dichlorophenyl)sulfonyl]amino}-2-pyridinyl)oxy]-N-[2-(dimethylamino)ethyl]-N-methylacetamide |
| 87 | 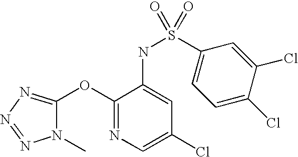 | Intermediate 56 | 437 | | 3,4-dichloro-N-{5-chloro-2-[(1-methyl-1H-tetrazol-5-yl)oxy]-3-pyridinyl}benzenesulfonamide |
| 88 | 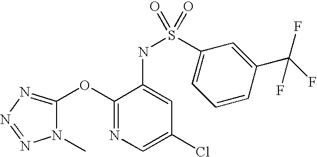 | Intermediate 56 | 435 | | N-{5-chloro-2-[(1-methyl-1H-tetrazol-5-yl)oxy]-3-pyridinyl}-3-(trifluoromethyl)benzene-sulfonamide |

### Example 89

### 3,4-dichloro-N-{5-chloro-2-[(4-methyl-2-pyridinyl)oxy]-3-pyridinyl}benzenesulfonamide

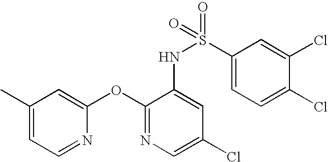

To a stirred solution of intermediate 45 (117 mg) in pyridine (3 ml) was added 3,4-dichlorobenzenesulfonyl chloride (93 uL) and DMAP (3 mg). The resultant mixture was heated to 70° C. for 2 hrs then cooled to RT and stirred for 72 hrs. Further 3,4-dichlorobenzenesulfonyl chloride (37 uL) was added and the resultant was stirred at 100° C. for a further 24 hours. The reaction was cooled and concentrated in vacuo and then loaded onto an isolute aminopropyl SPE. 3 volumes of methanol followed by 3 volumes of 2M ammonia in methanol were collected. The ammonia/methanol fractions were concentrated in vacuo to give a brown solid which was partitioned between ethyl aceate/water and separated. The ethyl acetate layer was washed with water, dried and concentrated in vacuo to afford the title compound 79 mg. MH+ 444/446.

### Example 90

### 3,4-dichloro-N-[5-chloro-2-(1H-1,2,4-triazol-3-ylamino)-3-pyridinyl]benzenesulfonamide

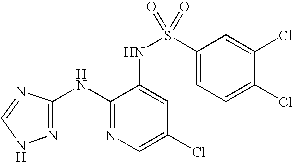

Intermediate 58 (18 mg, 0.086 mmol), 3,4-dichlorobenzenesulfonyl chloride (21 mg, 0.086 mmol), and pyridine (14 μL, 0.17 mmol) were dissolved in CHCl₃ and stirred at RT for 18 h. The reaction mixture was concentrated in vacuo and redisolved in DMSO. Purification was achieved using preparative HPLC (YMG 50×20 mm column, 20 mL/min flow rate, A: 0.1% TFA in acetonitrile B: 0.1% TFA in water, A: 20 to 100% over 10 min, UV detection at 215 nm) afforded the title compound as 4.8 mg (13% yield) of a tan solid. MH⁺419/421/423.

### Example 91

### 4-bromo-N-{5-chloro-2-[(2-methyl-3-pyridinyl)oxy]-3-pyridinyl}-2-methylbenzenesulfonamide

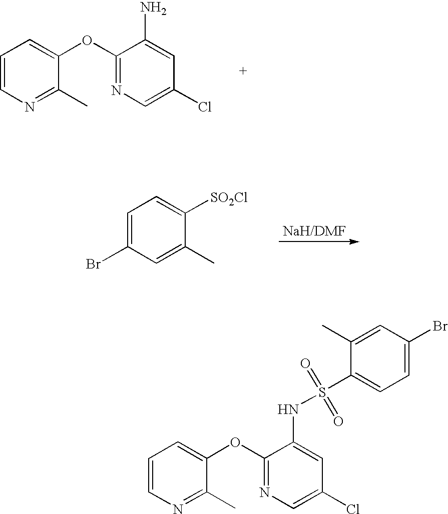

To a suspension of NaH (0.5 mmol) in DMF (1 mL) was added the solution of intermediate 33 in 1 mL DMF. After 10 min, the 4-bromo-2-methylbenzenesulfonyl chloride (0.25 mmol) was added and the mixture was stirred at 25° C. for 36 hours. The reaction was quenched with aq. NH₄Cl and extracted with EtOAc. The combined organic layers were washed with brine, dried with Na₂SO₄ and concentrated to provide 120 mg crude. The crude was purified with preparative HPLC to provide us yellow solid (5 mg, 4%). [MH]⁺=470

The following example was synthesized in an analogous manner to example 91 beginning with appropriate starting materials.

| Eg | Structure | From | +ve | −ve | Name |
|---|---|---|---|---|---|
| 92 | 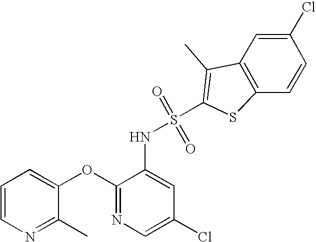 | Intermediate 33 | 480 | | 5-chloro-N-{5-chloro-2-[(2-methyl-3-pyridinyl)oxy]-3-pyridinyl}-3-methyl-1-benzothiophene-2-sulfonamide |

### Example 93

### N-{5-chloro-2-[(2-methyl-3-pyridinyl)oxy]-3-pyridinyl}-4-(1,1-dimethylpropyl)benzenesulfonamide

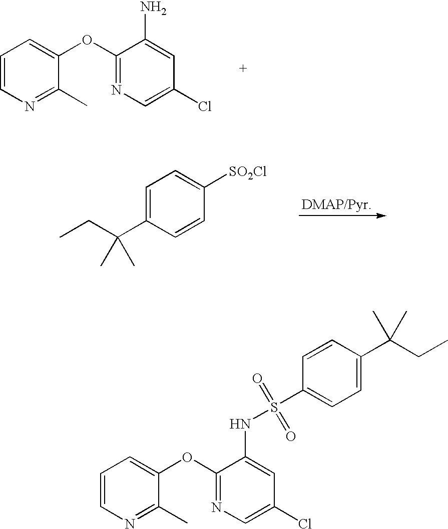

To a solution of the sulfonyl chloride (0.6 mmol), DMAP (12 mg, 0.1 mmol) in pyridine (2 mL) was stirred for 2 min. Then the solution of intermediate 33 in 1 mL pyridine was added and the mixture was stirred at 90° C. for 3 hours. The reaction was cooled and poured 15 mL water. The mixture was extracted with EtOAc, the combined organic layers were washed with brine, dried with Na₂SO₄ and concentrated to provide 140 mg crude. The crude was purified with preparative HPLC to provide off-white solid (35 mg, 18%). [MH]⁺=446.

The following example was synthesized in an analogous manner to example 93.

| Eg | Structure | From | +ve | −ve | Name |
|---|---|---|---|---|---|
| 94 | 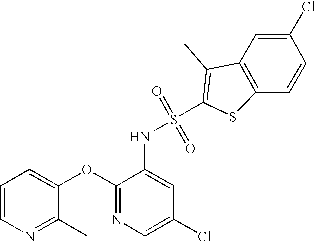 | Intermediate 33 | 480 | | 5-chloro-N-{5-chloro-2-[(2-methyl-3-pyridinyl)oxy]-3-pyridinyl}-3-methyl-1-benzothiophene-2-sulfonamide |
| 95 | 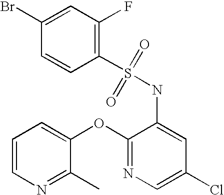 | Intermediate 33 | 474 | | 4-bromo-N-{5-chloro-2-[(2-methyl-3-pyridinyl)oxy]-3-pyridinyl}-2-fluorobenzenesulfonamide |
| 96 | 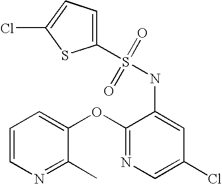 | Intermediate 33 | 416 | | 5-chloro-N-{5-chloro-2-[(2-methyl-3-pyridinyl)oxy]-3-pyridinyl}-2-thiophenesulfonamide |
| 97 | 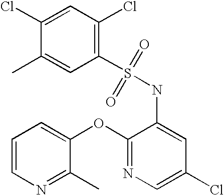 | Intermediate 33 | 460 | | 2,4-dichloro-N-{5-chloro-2-[(2-methyl-3-pyridinyl)oxy]-3-pyridinyl}-5-methylbenzene-sulfonamide |
| 98 | 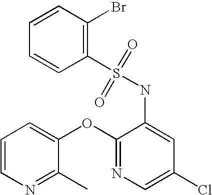 | Intermediate 33 | 456 | | 2-bromo-N-{5-chloro-2-[(2-methyl-3-pyridinyl)oxy]-3-pyridinyl}benzene-sulfonamide |
| 99 | 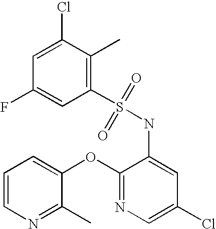 | Intermediate 33 | 442 | | 3-chloro-N-{5-chloro-2-[(2-methyl-3-pyridinyl)oxy]-3-pyridinyl}-5-fluoro-2-methylbenzene-sulfonamide |
| 100 | 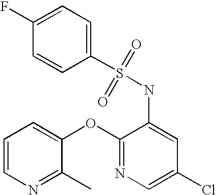 | Intermediate 33 | 394 | | N-{5-chloro-2-[(2-methyl-3-pyridinyl)oxy]-3-pyridinyl}-4-fluorobenzene-sulfonamide |
| 101 | 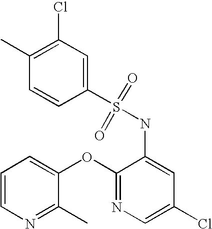 | Intermediate 33 | 424 | | 3-chloro-N-{5-chloro-2-[(2-methyl-3-pyridinyl)oxy]-3-pyridinyl}-4-methylbenzene-sulfonamide |
| 102 | 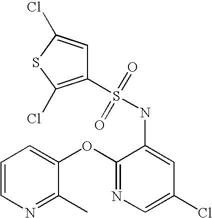 | Intermediate 33 | 451 | | 2,5-dichloro-N-{5-chloro-2-[(2-methyl-3-pyridinyl)oxy]-3-pyridinyl}-3-thiophenesulfonamide |

### Example 103

### 2-[(5-chloro-3-{[(3,4-dichlorophenyl)sulfonyl]amino}-2-pyridinyl)oxy]-N-(2-hydroxyethyl)benzamide

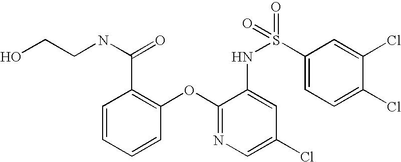

To a stirred solution of the intermediate 75 (515 mg) in anhydrous DMF (5 mL) was added ethanolamine (138 mg) in 1 portion. The solution darkened immediately upon addition before been heated to 40° C. for 15 minutes. The crude reaction mixture was cooled and concentrated in vacuo and loaded onto an isolute aminopropyl ion-exchange SPE. Eluting with methanol (3 volumes) and 2M ammonia in methanol (3 volumes). The ammonia methanol fractions were concentrated in vacuo to give a solid which was partitioned between water and chloroform, eluted through a phase separator and concentrated to give an orange solid.

The orange solid was purified further by silica gel SPE (1-5% Methanol in DCM) to afford the title compound. 181 mg. MH+518

### Example 104

### 2-[(5-chloro-3-{[(4,5-dichloro-2-thienyl)sulfonyl]amino}-2-pyridinyl)oxy]-N-methylbenzamide

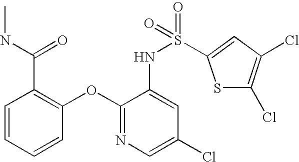

To a suspension of sodium hydride (60% dispersion in mineral oil, 57 mg) in anhydrous THF was added intermediate 74 (177 mg) in 1 portion. The resultant purple/brown solution was stirred at 0° C. for 15 mins. 4,5-dichloro-2-thiophenesulfonyl chloride (216 mg) was then added and the reaction mixture was stirred for 60 minutes at RT. 2M hydrochloric acid (4 mL) was added drop-wise and the reaction was concentrated in vacuo to give orange solid.

The orange solid was dissolved in anhydrous DMF (10 mL) and methylamine (44 mg) was added. The resultant was stirred at room temperature for 72 hours before been concentrated in vacuo to afford an orange oil. Purification using MDAP to afforded the title compound 24 mg. MH+ 494.

The following examples were prepared by a method analogous to that described above for Example 104 from intermediate 74 using appropriate starting materials.

| Eg | Structure | From | +ve | −ve | Name |
|---|---|---|---|---|---|
| 105 | 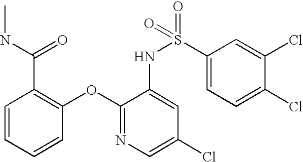 | Intermediate 74 | 444 | 442 | N-{5-chloro-2-[(2-methyl-3-pyridinyl)oxy]-3-pyridinyl}-3-(trifluoromethyl)-benzenesulfonamide |
| 106 | 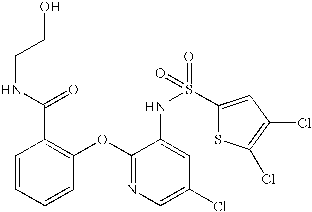 | Intermediate 74 | 488 | 486 | 2-[(5-chloro-3-{[(4,5-dichloro-2-thienyl)sulfonyl]amino}-2-pyridinyl)oxy}-N-(2-hydroxyethyl)benzamide |

### Example 107

### 2-[(3-{[(4-chlorophenyl)sulfonyl]amino}-2-pyridinyl)oxy]benzoic acid

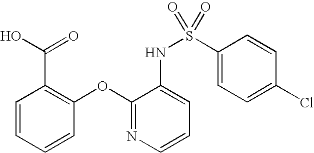

Intermediate 30, (500 mg), 4-chlorobenzenesulfonyl chloride (432 mg) and 4-dimethylaminopyridine (20 mg) were dissolved in pyridine (10 mL) and heated at reflux for 1 hour. The pyridine was removed in vacuo and the residue purified by column chromatography and the residue triturated with 2-propanol. The resulting solid was heated at reflux for 1 hour in methanol (8 mL) and water (2 mL) in the presence of lithium hydroxide hydrate (120 mg). 2M HCl (10 mL) was added and the mixture filtered and dried to provide the title compound (210 mg) as a white solid. [MH]⁺=405

### Example 108

### 2-[(3-{[(3,4-dichlorophenyl)sulfonyl]-amino}-2-pyridinyl)oxy]benzoic acid

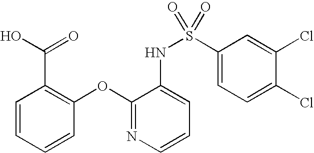

Example 108 was prepared in a manner analogous to that described above for example 107 from intermediate 30 and 3,4-dichlorobenzenesulfonyl chloride. [MH]⁺=439; ¹H NMR (DMSO-d₆) δ 12.67 (br.s, 1H), 10.48 (br.s, 1H), 8.04 (br.s, 1H), 7.85 (m, 3H), 7.78 (m, 1H), 7.73 (m, 3H), 7.55 (t, 1H), 7.31 (t, 1H), 7.04 (dd, 1H), 6.70 (dd, 1H).

### Example 109

### 2-[(5-chloro-3-{[(4-chlorophenyl)sulfonyl]amino}-2-pyridinyl)oxy]benzoic acid

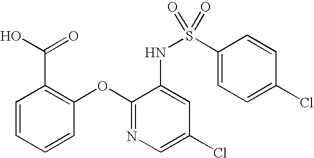

Example 109 was prepared in a manner analogous to that described above for example 107 from intermediate 29 and 4-chlorobenzenesulfonyl chloride; HPLC m/z [MH]⁺=439

### Example 110

### 2-[(5-chloro-3-{[(4-chlorophenyl)sulfonyl]amino}-2-pyridinyl)oxy]-N-methylbenzamide

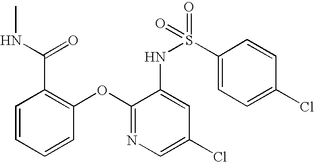

To a stirred solution of example 109 (84 mg) in anhydrous DCM (5 ml) was sequentially added DIPEA (di-i-propylethylamine) (36 ul), TBtU (67 mg) and 2M methylamine in THF (287 ul). The resultant was stirred for 18 hours at RT. A further 2M methylamine in THF (287 ul) and TBtu (40 mg) was added and the reaction was stirred for 30 minutes. The solvents were removed in vacuo to give a yellow oil. The oil was purified initially by silica gel SPE (20-80% ethyl acetate in hexane) to afford a yellow oil. Further purification was accomplished by MDAP to afford the title compound. 13 mg (15%); MH+ 452.

### Example 111

### 2-{[3-{[(4-chlorophenyl)sulfonyl]amino}-6-(methyloxy)-2-pyridinyl]oxy}benzoic acid

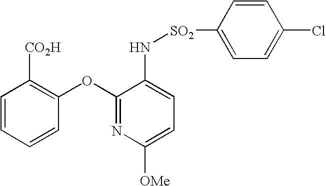

Ethyl salicylate (199 mg) was dissolved in DMF. To this was added potassium carbonate (207 mg) and 2-chloro-6-(methyloxy)-3-nitropyridine (188 mg). The mixture was heated in a microwave reactor at 120° C. for 30 minutes and then the solvent removed in vacuo. The residue was partitioned between DCM (10 mL) and aqueous 1N sodium hydroxide solution (5 mL) and the organic phase was concentrated under reduced pressure. This was dissolved in ethanol (10 mL) and 10% palladium on carbon added (5 mg). The mixture was stirred under an atmosphere of hydrogen at room temperature for 12 hours. The slurry was filtered through Celite® and the filtrate concentrated under reduced pressure and dissolved in pyridine (3 mL). To this was added 4-chlorobenzene sulfonyl chloride (422 mg). The mixture was heated in a microwave reactor at 120° C. for 30 minutes and then the volatiles removed in vacuo. The residue was partitioned between DCM (20 mL) and aqueous 1N sodium hydroxide (5 mL). The organic phase was concentrated under reduced pressure, dissolved in 2:1 tetrahydrofuran:water and lithium hydroxide (24 mg) was added. The mixture was heated to 125° C. in a microwave reactor for 30 minutes and then the solvent removed in vacuo. The residue was purified by preparative HPLC to provide the title compound. [MH]⁺=435, [M]⁻=433.

### Example 112

### 2-[(5-chloro-3-{[(3,4-dichlorophenyl)sulfonyl]amino}-2-pyridinyl)oxy]-N-[2-(1H-imidazol-4-yl)ethyl]benzamide

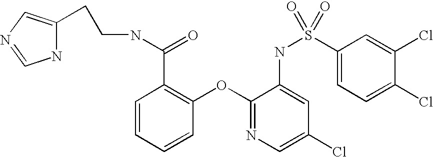

To a suspension of histamine dihydrochloride (17 mg) in DMF (0.45 ml) was added N,N-dimethylaminopyridine (2 mg), N-(3-dimethylaminopropyl)-N′-ethylcarbodiimide (29 mg), and 2-[(5-chloro-3-{[(3,4-dichlorophenyl)sulfonyl]amino}-2-pyridinyl)oxy]benzoic acid (64 mg, example 1). The solution was stirred for 12 h at room temperature. The solution was then diluted with methanol (5 ml) and filtered through an aminopropyl-SPE column. The product was eluted with 5% ammonia/methanol and purified by auto-preparative HPLC. [MH]⁺=568.

The following examples were prepared from 2-[(5-chloro-3-{[(3,4-dichlorophenyl)sulfonyl]amino}-2-pyridinyl)oxy]benzoic acid, 3-[(5-chloro-3-{[(3,4-dichlorophenyl)sulfonyl]amino}-2-pyridinyl)oxy]benzoic acid, or 4-[(5-chloro-3-{[(3,4-dichlorophenyl)sulfonyl]amino}-2-pyridinyl)oxy]benzoic acid by a method analogous to that described above for example 1 with the appropriate amine followed by purification using aminopropyl-SPE and/or auto-preparative HPLC. In some examples, heating to 60° C. was required.

| Eg | Structure | [MH]⁺ | [MH]⁻ | Name |
|---|---|---|---|---|
| 113 | 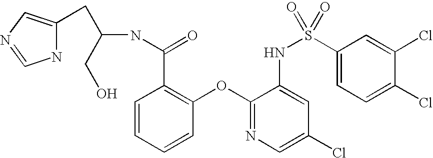 | 598 | | 2-[(5-chloro-3-{[(3,4-dichlorophenyl)sulfonyl]amino}-2-pyridinyl)oxy]-N-[2-hydroxy-1-(1H-imidazol-4-ylmethyl)ethyl]benzamide |
| 114 | 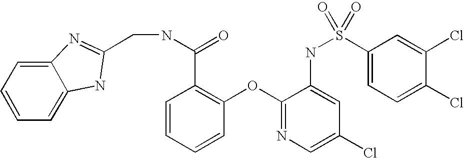 | 604 | | N-(1H-benzimidazol-2-ylmethyl)-2-[(5-chloro-3-{[(3,4-dichlorophenyl)sulfonyl]amino}-2-pyridinyl)oxy]benzamide |
| 115 | 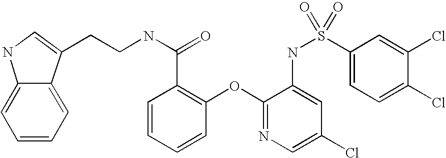 | 617 | | 2-[(5-chloro-3-{[(3,4-dichlorophenyl)sulfonyl]amino}-2-pyridinyl)oxy]-N-[2-(1H-indol-3-yl)ethyl]benzamide |
| 116 | 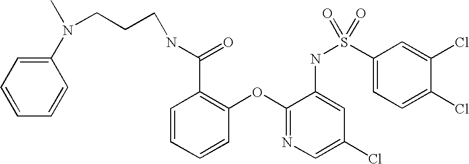 | 621 | | 2-[(5-chloro-3-{[(3,4-dichlorophenyl)sulfonyl]amino}-2-pyridinyl)oxy]-N-{3-[methyl(phenyl)amino]-propyl}-benzamide |
| 117 | 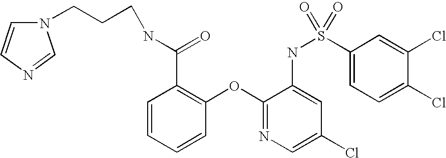 | 582 | | 2-[(5-chloro-3-{[(3,4-dichlorophenyl)sulfonyl]amino}-2-pyridinyl)oxy]-N-[3-(1H-imidazol-1-yl)propyl]benzamide |
| 118 | 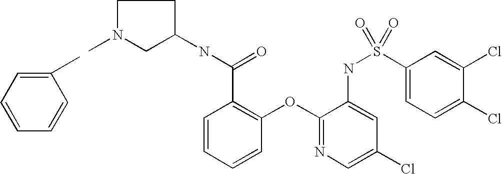 | 633 | | 2-[(5-chloro-3-{[(3,4-dichlorophenyl)sulfonyl]amino}-2-pyridinyl)oxy]-N-[1-(phenylmethyl)-3-pyrrolidinyl]benzamide |
| 119 | 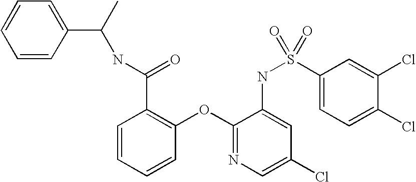 | 578 | | 2-[(5-chloro-3-{[(3,4-dichlorophenyl)sulfonyl]amino}-2-pyridinyl)oxy]-N-[(1S)-1-phenylethyl]benzamide |
| 120 | 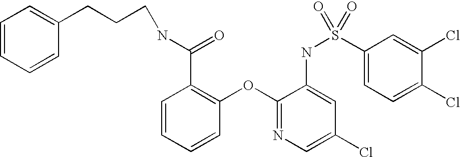 | 592 | | 2-[(5-chloro-3-{[(3,4-dichlorophenyl)sulfonyl]amino}-2-pyridinyl)oxy]-N-(3-phenylpropyl)benzamide |
| 121 | 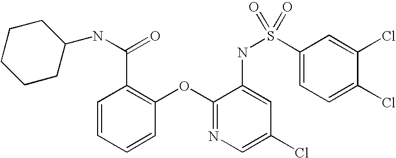 | 556 | | 2-[(5-chloro-3-{[(3,4-dichlorophenyl)sulfonyl]amino}-2-pyridinyl)oxy}-N-cyclohexylbenzamide |
| 122 | 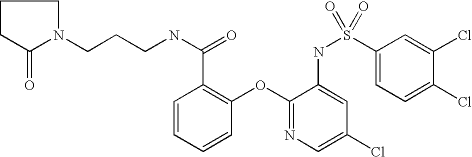 | 599 | | 2-[(5-chloro-3-{[(3,4-dichlorophenyl)sulfonyl]amino}-2-pyridinyl)oxy]-N-[3-(2-oxo-1-pyrrolidinyl)propyl}benzamide |
| 123 | 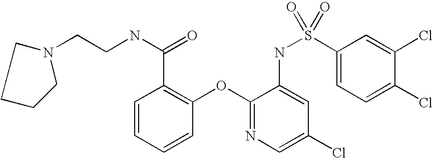 | 571 | | 2-[(5-chloro-3-{[(3,4-dichlorophenyl)sulfonyl]amino}-2-pyridinyl)oxy]-N-[2-(1-pyrrolidinyl)ethyl]benzamide |
| 124 | 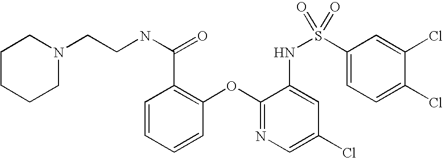 | 585 | | 2-[(5-chloro-3-{[(3,4-dichlorophenyl)sulfonyl]amino}-2-pyridinyl)oxy]-N-(2-(1-piperidinyl)ethyl]benzamide |
| 125 | 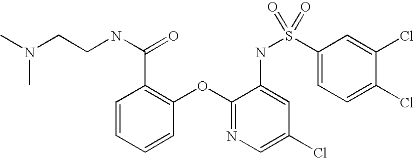 | 545 | | 2-[(5-chloro-3-{[(3,4-dichlorophenyl)sulfonyl]amino}-2-pyridinyl)oxy]-N-[2-(dimethylamino)ethyl]benzamide |
| 126 | 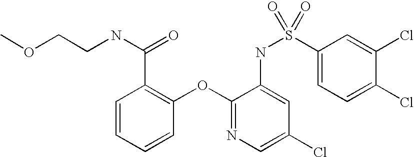 | 532 | | 2-[(5-chloro-3-{[(3,4-dichlorophenyl)sulfonyl]amino}-2-pyridinyl)oxy]-N-[2-(methyloxy)ethyl]benzamide |
| 127 | 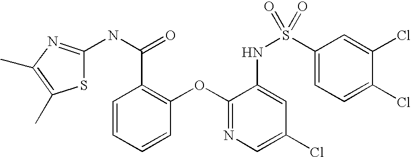 | 585 | | 2-[(5-chloro-3-{[(3,4-dichlorophenyl)sulfonyl]amino}-2-pyridinyl)oxy]-N-(4,5-dimethyl-1,3-thiazol-2-yl)benzamide |
| 128 | 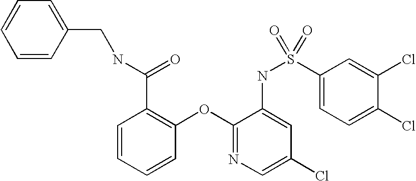 | 564 | | 2-[(5-chloro-3-{[(3,4-dichlorophenyl)sulfonyl]amino}-2-pyridinyl)oxy]-N-(phenylmethyl)benzamide |
| 129 | 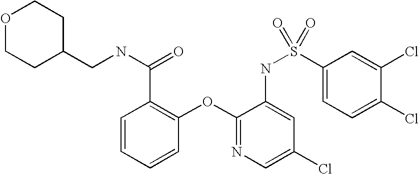 | 572 | | 2-[(5-chloro-3-{[(3,4-dichlorophenysulfonyl]amino}-2-pyridinyl)oxy]-N-(tetrahydro-2H-pyran-4-ylmethyl)benzamide |
| 130 | 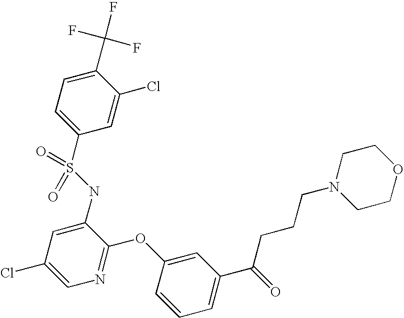 | | 618 | 3-{[5-chloro-3-({[3-chloro-4-(trifluoromethyl)phenyl]-sulfonyl}amino)-2-pyridinyl]oxy}-N-[2-(4-morpholinyl)ethyl]benzamide |
| 131 | 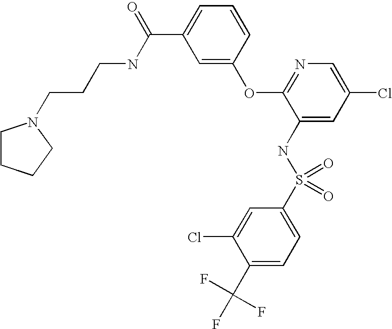 | | 616 | 3-{[5-chloro-3-({[3-chloro-4-(trifluoromethyl)phenyl]-sulfonyl}amino)-2-pyridinyl]oxy}-N-[3-(1-pyrrolidinyl)propyl]benzamide |
| 132 | 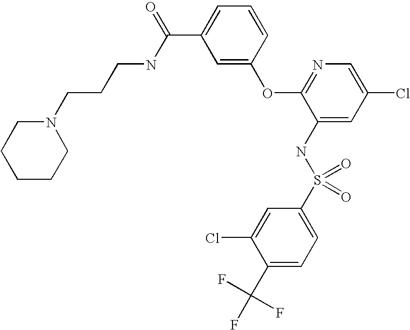 | | 630 | 3-{[5-chloro-3-({[3-chloro-4-(trifluoromethyl)phenyl]-sulfonyl}amino)-2-pyridiny))oxy}-N-[3-(1-piperidinyl)propyl]benzamide |
| 133 | 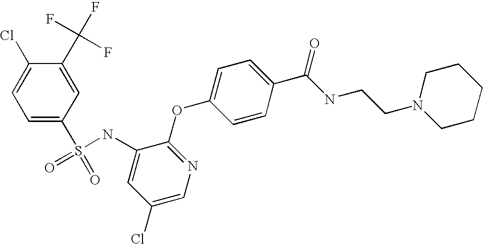 | | 617 | 4-{[5-chloro-3-({[4-chloro-3-(trifluoromethyl)phenyl]-sulfonyl}amino)-2-pyridinyl]oxy}-N-[2-(1-piperidinyl)ethyl]benzamide |
| 134 | 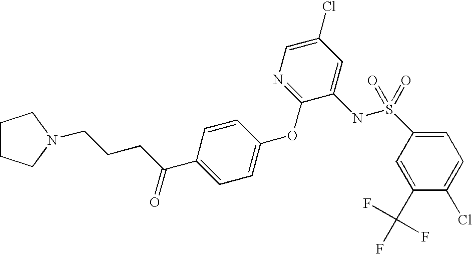 | | 603 | 4-{[5-chloro-3-({[4-chloro-3-(trifluoromethyl)phenyl]-sulfonyl}amino)-2-pyridinyl]oxy}-N-[2-(1-pyrrolidinyl)ethyl]benzamide |
| 135 | 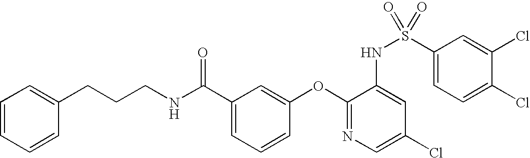 | 591 | | 3-[(5-chloro-3-{[(3,4-dichlorophenyl)sulfonyl]amino}-2-pyridinyl)oxy]-N-(3-phenylpropyl)benzamide |
| 136 | 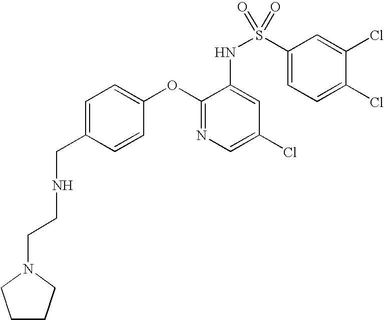 | 633 | | 3-[(5-chloro-3-{[(3,4-dichlorophenyl)sulfonyl]amino}-2-pyridinyl)oxy]-N-{(3S)-1-(phenylmethyl)-3-pyrrolidinyl]benzamide |
| 137 | 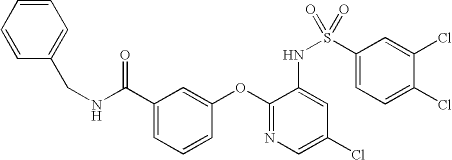 | 564 | | 3-[(5-chloro-3-{[(3,4-dichlorophenyl)sulfonyl]amino}-2-pyridinyl)oxy]-N-(phenylmethyl)benzamide |
| 138 | 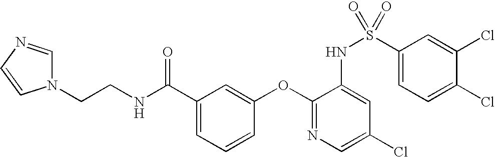 | 582 | | 3-[(5-chloro-3-{[(3,4-dichlorophenyl)sulfonyl]amino}-2-pyridinyl)oxy]-N-13-(1H-imidazol-1-yl)propyl]benzamide |
| 139 | 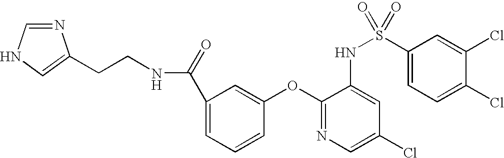 | 568 | | 3-[(5-chloro-3-{[(3,4-dichlorophenyl)sulfonyl]amino}-2-pyridinyl)oxy]-N-[2-(1H-imidazol-4-yl)ethyl]benzamide |
| 140 | 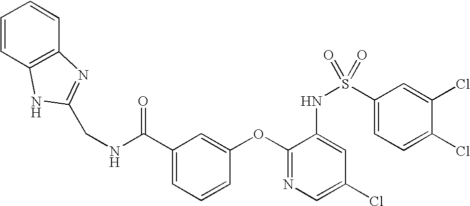 | 604 | | N-(1H-benzimidazol-2-ylmethyl)-3-[(5-chloro-3-{[(3,4-dichlorophenyl)sulfonyl]amino}-2-pyridinyl)oxy]benzamide |
| 141 | 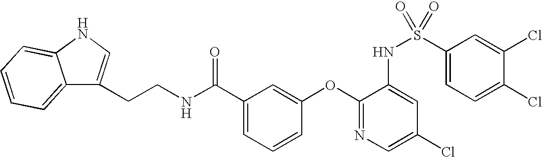 | 617 | | 3-[(5-chloro-3-{[(3,4-dichlorophenyl)sulfonyl]amino}-2-pyridinyl)oxy]-N-[2-(1H-indol-3-yl)ethyl]benzamide |
| 142 | 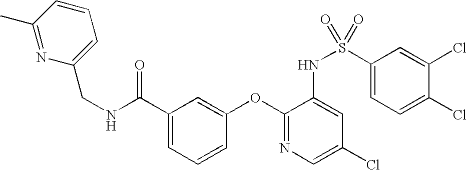 | 579 | | 3-[(5-chloro-3-{[(3,4-dichlorophenyl)sulfonyl]amino}-2-pyridinyl)oxy]-N-[(6-methyl-2-pyridinyl)methyl]benzamide |
| 143 | 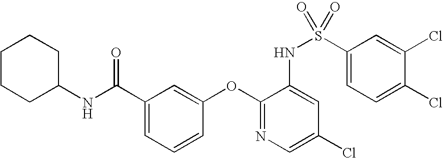 | 556 | | 3-[(5-chloro-3-{[(3,4-dichlorophenyl)sulfonyl]amino}-2-pyridinyl)oxy]-N-cyclohexylbenzamide |
| 144 | 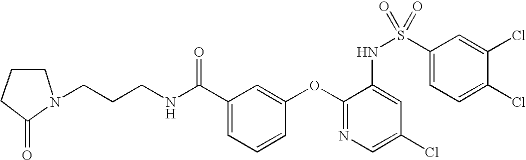 | 599 | | 3-[(5-chloro-3-{[(3,4-dichlorophenyl)sulfonyl]amino}-2-pyridinyl)oxy]-N-[3-(2-oxo-1-pyrrolidinyl)propyl]benzamide |
| 145 | 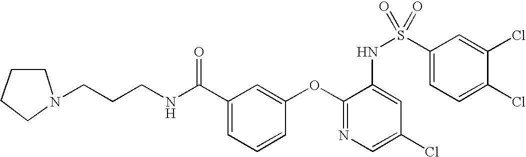 | 571 | | 3-[(5-chloro-3-{[(3,4-dichlorophenyl)sulfonyl]amino}-2-pyridinyl)oxy]-N-[2-(1-pyrrolidinyl)ethylbenzamide |
| 146 | 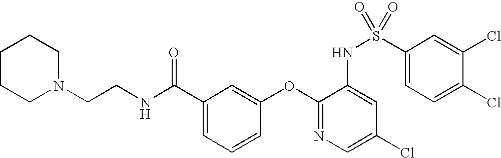 | 585 | | 3-[(5-chloro-3-{[(3,4-dichlorophenyl)sulfonyl]amino}-2-pyridinyl)oxy]-N-[2-(1-piperidinyl)ethyl]benzamide |
| 147 | 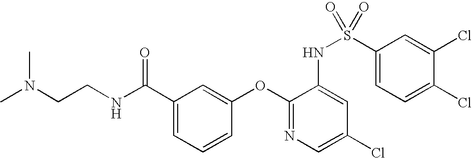 | 545 | | 3-[(5-chloro-3-{[(3,4-dichlorophenyl)sulfonyl]amino}-2-pyridinyl)oxy]-N-[2-(dimethylamino)ethyl]benzamide |
| 148 | 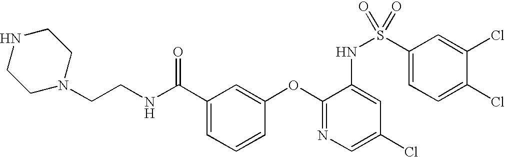 | 586 | | 3-[(5-chloro-3-{[(3,4-dichlorophenyl)sulfonyl]amino}-2-pyridinyl)oxy]-N-[2-(1-piperazinyl)ethyl]benzamide |
| 149 | 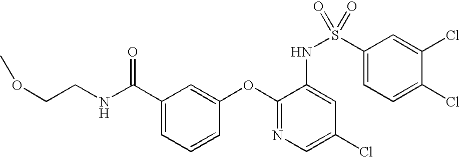 | 532 | | 3-[(5-chloro-3-{[(3,4-dichlorophenyl)sulfonyl]amino}2-pyridinyl)oxy]-N-[2-(methyloxy)ethyl]benzamide |
| 150 | 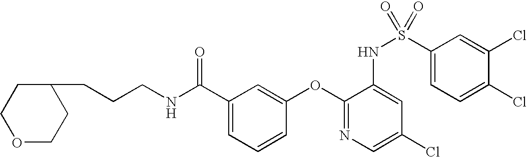 | 601 | | 3-[(5-chloro-3-{[(3,4-dichlorophenyl)sulfonyl]amino}-2-pyridinyl)oxy]-N-[3-(4-morpholinyl)propyl]benzamide |
| 151 | 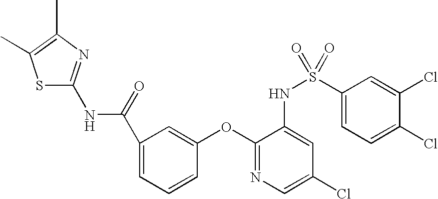 | 585 | | 3-[(5-chloro-3-{[(3,4-dichlorophenyl)sulfonyl]amino}-2-pyridinyl)oxy]-N-(4,5-dimethyl-1,3-thiazoi-2-yl)benzamide |
| 152 | 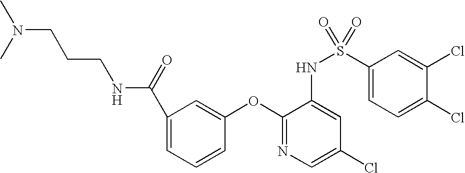 | 559 | | 3-{(5-chloro-3-{[(3,4-dichlorophenyl)sulfonyl]amino}-2-pyridinyl)oxy]-N-[3-(dimethylamino)-propyl]benzamide |
| 153 | 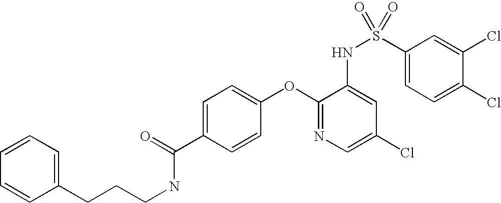 | 592 | | 4-[(5-chloro-3-{[(3,4-dichlorophenyl)sulfonyl]amino}-2-pyridinyl)oxy]-N-(3-phenylpropyl)benzamide |
| 154 | 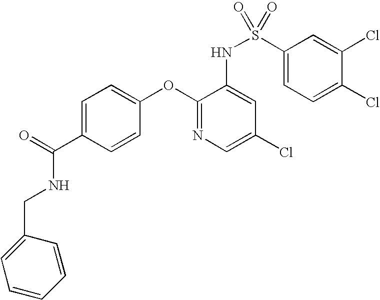 | 564 | | 4-[(5-chloro-3-{[(3,4-dichlorophenyl)sulfonyl]amino}-2-pyridinyl)oxy]-N-(phenylmethyl)benzamide |
| 155 | 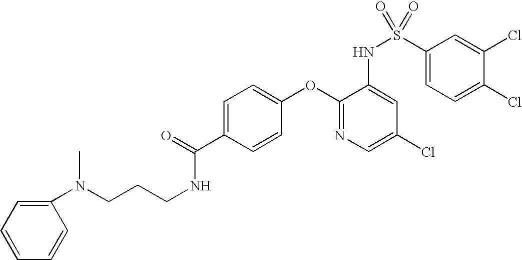 | 621 | | 4-[(5-chloro-3-{[(3,4-dichlorophenyl)sulfonyl]amino}-2-pyridinyl)oxy]-N-{3-[methyl(phenyl)amino]-propyl}benzamide |
| 156 | 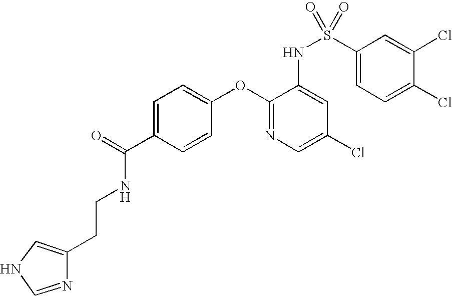 | 568 | | 4-[(5-chloro-3-{[(3,4-dichlorophenyl)sulfonyl]amino}-2-pyridinyl)oxy]-N-[2-(1H-imidazol-4-ethyl]benzamide |
| 157 | 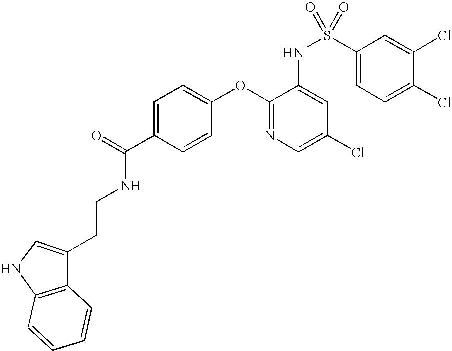 | 617 | | 4-[(5-chloro-3-{[(3,4-dichlorophenyl)sulfonyl]amino}-2-pyridinyl)oxy]-N-[2-(1H-indol-3-yl)ethyl]benzamide |
| 158 | 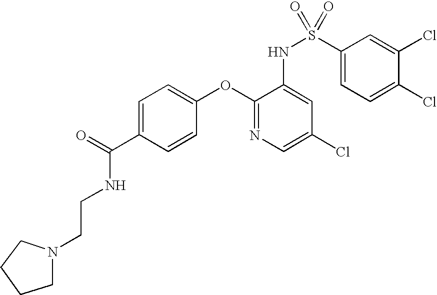 | 571 | | 4-[(5-chloro-3-{[(3,4-dichlorophenyl)sulfonyl]amino}-2-pyridinyl)oxy]-N-[2-(1-pyrrolidinyl)ethyl]benzamidetrifluoroacetate |
| 159 | 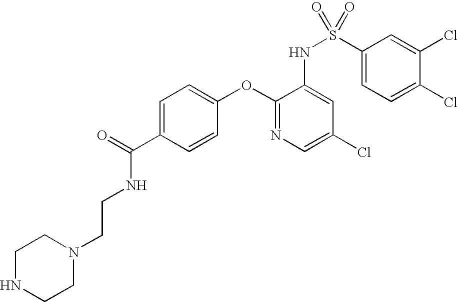 | 586 | | 4-[(5-chloro-3-{[(3,4-dichlorophenyl)sulfonyl]amino}-2-pyridinyl)oxy]-N-[2-(1-piperazinyl)ethyl]benzamidetrifluoroacetate |
| 160 | 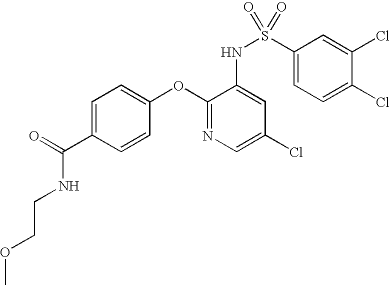 | 532 | | 4-[(5-chloro-3-{[(3,4-dichlorophenyl)sulfonyl]amino}-2-pyridinyl)oxy]-N-[2-(methyloxy)ethyl]benzamidetrifluoroacetate |
| 161 | 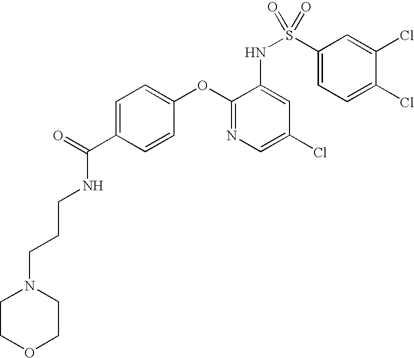 | 601 | | 4-[(5-chloro-3-{[(3,4-dichlorophenyl)sulfonyl]amino}-2-pyridinyl)oxy]-N-{3-(4-morpholinyl)propyl]benzamidetrifluoroacetate |
| 162 | 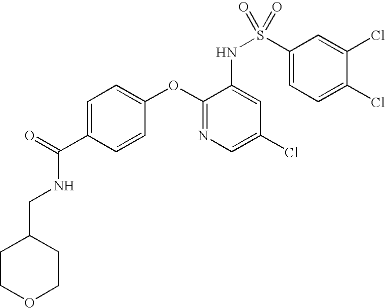 | 572 | | 4-[(5-chloro-3-{[(3,4-dichlorophenyl)sulfonyl]amino}-2-pyridinyl)oxy]-N-(tetrahydro-2H-pyran-4-ylmethyl)benzamidetrifluoroacetate |
| 163 | 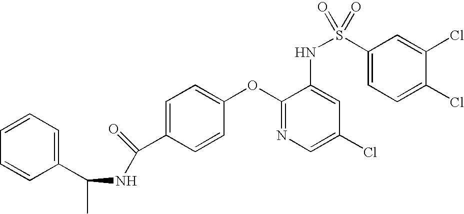 | 578 | | 4-[(5-chloro-3-{[(3,4-dichlorophenyl)sulfonyl]amino}-2-pyridinyl)oxy]-N-[(1R)-1-phenylethyl]benzamidetrifluoroacetate |

### Example 164

### 3,4-dichloro-N-(5-chloro-2-{[4-(1H-tetrazol-5-yl)phenyl]oxy}-3-pyridinyl)benzenesulfonamide

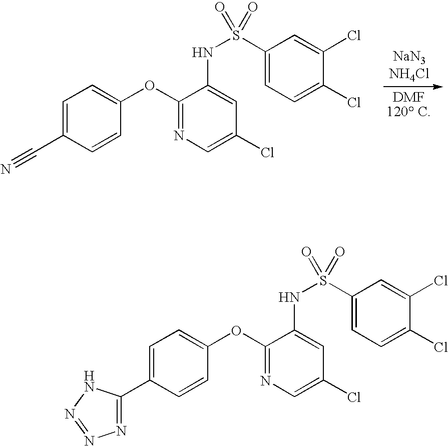

The reaction mixture of 3,4-dichloro-N-{5-chloro-2-[(4-cyanophenyl)oxy]-3-pyridinyl}benzenesulfonamide (33 mg, 0.07 mmol), sodium azide (23 mg, 0.36 mmol) and ammonium chloride (19 mg, 0.36 mmol) in anhydrous DMF (2 mL) was stirred at 120° C. for 12 hours. After removal of the solvent, the residue was suspended in water (3 mL), basified with 5% NaOH, and washed with Et₂O. The aqueous layer was acidified to pH 3 with 10% HCl and extracted with ethyl acetate, the organic layer was washed with water, dried (MgSO₄), and concentrated to give a crude product, which was purified by column chromatography on silica gel, the desired compound was obtained as a white solid (22 mg, 61%). HPLC Rt=2.98 minutes, m/z [MH]⁺=498. ¹H NMR (MeOD) δ 8.01 (s, 1H), 7.98 (s, 1H), 7.96 (d, 1H), 7.93 (d, 1H), 7.85 (d, 1H), 7.64 (m, 2H), 6.84 (m, 2H). Proceeding in a similar manner, but replacing 3,4-dichloro-N-{5-chloro-2-[(4-cyanophenyl)oxy]-3-pyridinyl}benzenesulfonamide with 3,4-dichloro-N-{5-chloro-2-[(3-cyanophenyl)oxy]-3-pyridinyl}benzenesulfonamide, the compound example 166 listed in the table below was prepared.

| Eg | Structure | +ve | −ve | Name |
|---|---|---|---|---|
| 165 | 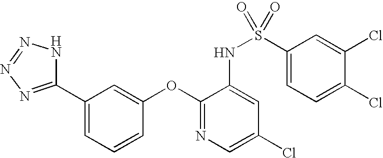 | 499 | | 3,4-dichloro-N-(5-chloro-2-{[3-(1H-tetrazol-5-yl)phenyl]loxy}-3-pyridinyl)benzenesulfonamide |

### Example 166

### 3,4-dichloro-N-{5-chloro-2-[(4-{[(phenylmethyl)amino]methyl}phenyl)oxy]-3-pyridinyl}benzenesulfonamide

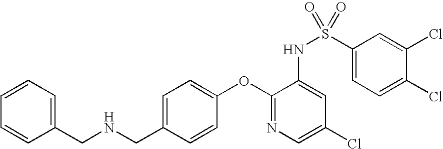

Intermediate 76 (49.8 mgl) was dissolved in anhydrous methylene chloride (0.5 mL) in a vial then benzyl amine (16.0 mg) was added with TEA (0.02 mL) and glacial AcOH (0.004 mL). The mixture was allowed to stir at room temperature for 30 min. then sodium triacetoxy borohydride (46.2 mg) was added. The reaction stirred overnight at room temperature and upon completion the reactions were quenched with saturated sodium bicarbonate solution. The layers were separated and washed with sodium bicarbonate solution, water and brine. The organic layer was concentrated and purified by acidic Gilson HPLC. [MH]⁺=549.

The following examples were made in a manner analogous to that for example 2 beginning with either 3,4-dichloro-N-{5-chloro-2-[(3-formylphenyl)oxy]-3-pyridinyl}benzenesulfonamide or 3,4-dichloro-N-{5-chloro-2-[(4-formylphenyl)oxy]-3-pyridinyl}benzenesulfonamide using the appropriate amine.

| | | | | |
|---|---|---|---|---|
| Eg | Structure | +ve | −ve | Name |

| | | | |
|---|---|---|---|
| 167 | 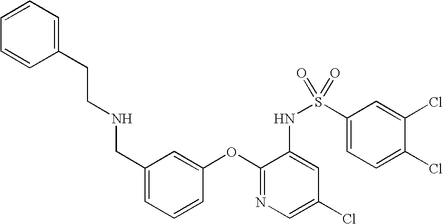 | 564 | 3,3-dichloro-N-{5-chloro-2-[(3-{[(2-phenylethyl)amino)methyl}phenyl)oxy]-3-pyridinyl}benzenesulfonamidetrifluoroacetate |
| 168 | 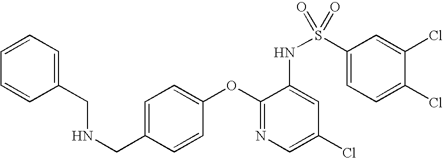 | 550 | 3,4-dichloro-N-{5-chloro-2-{(4-{[(phenylmethyl)amino]methyl}phenyl)oxy]-3-pyridinyl}benzenesulfonamidetrifluoroacetate |
| 169 | 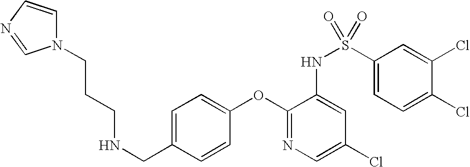 | 568 | 3,4-dichloro-N-(5-chloro-2-{[4-({[3-(1H-imidazol-1-yl)propyl]amino}methyl)phenyl]oxy}-3-pyridinyl)benzenesulfonamidetrifluoroacetate |
| 170 | 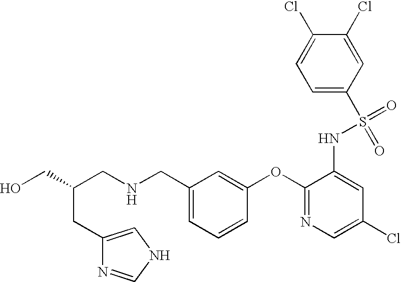 | 598 | 3,4-dichloro-N-(5-chloro-2-{[3-({[(2S)-3-hydroxy-2-(1H-imidazol-4-ylmethyl)propyl]amino}methyl)phenyl]oxy}-3-pyridinyl)benzenesulfonamidetrifluoroacetate (salt) |
| 171 | 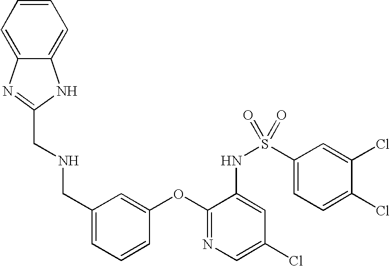 | 590 | N-{2-{[(3-{[(1H-benzimidazol-2-ylmethyl)amino]methyl}phenyl)oxy]-5-chloro-3-pyridinyl}-3,4-dichlorobenzenesulfonamidetrifluoroacetate |
| 172 | 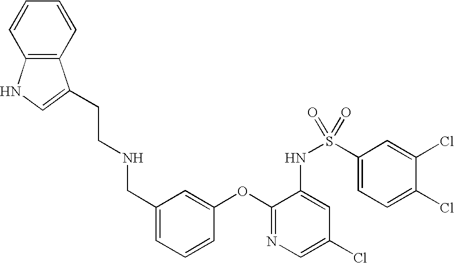 | 603 | 3,4-dichloro-N-(5-chloro-2-{[3-({[2-(1H-indol-3-yl)ethyl]amino}methyl)phenyl]oxy}-3-pyridinyl)benzenesulfonamidetrifluoroacetate |
| 173 | 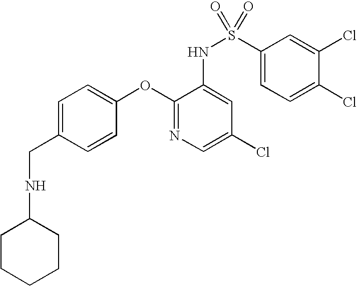 | 542 | 3,4-dichloro-N-[5-chloro-2-({4-[(cyclohexylamino)methyl]phenyl}oxy)-3-pyridinyl]benzenesulfonamidetrifluoroacetate |
| 174 | 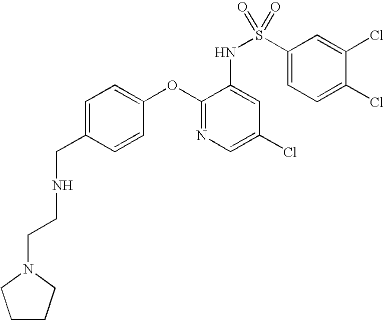 | 557 | 3,4-dichloro-N-(5-chloro-2-{[4-({[2-(1-pyrrolidinyl)ethyl]-amino}methyl)phenyl]oxy}-3-pyridinyl)benzenesulfonamidetrifluoroacetate |
| 175 | 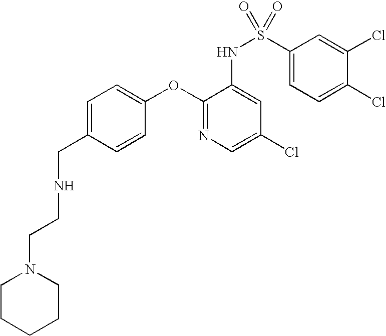 | 571 | 3,4-dichloro-N-(5-chloro-2-{[4-({[2-(1-piperidinyl)ethyl]-amino}methyl)phenyl]oxy}-3-pyridinyl)benzenesulfonamidetrifluoroacetate |
| 176 | 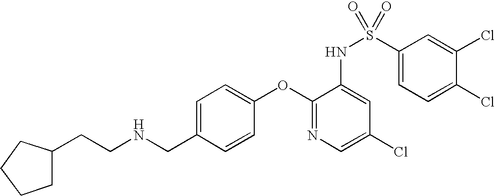 | 557 | 3,4-dichloro-N-(5-chloro-2-{[4-({[2-(1-pyrrolidinyl)ethyl]-amino}methyl)phenyl]oxy}-3-pyridinyl)benzenesulfonamide |
| 177 | 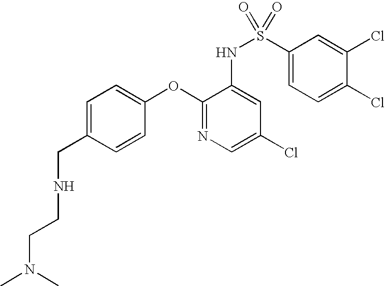 | 531 | 3,4-dichloro-N-(5-chloro-2-{[4-({[2-(dimethylamino)ethyl]-amino}methyl)phenyl]-oxy}-3-pyridinyl)benzenesulfonamidetrifluoroacetate |
| 178 | 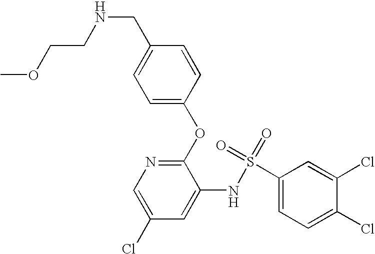 | 518 | 3,4-dichloro-N-(5-chloro-2-{[4-({[2-(methyloxy)ethyl]-amino}methyl)phenyl]oxy}-3-pyridinyl)benzenesulfonamidetrifluoroacetate |
| 179 | 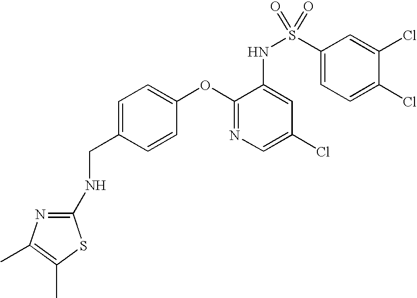 | 571 | 3,4-dichloro-N-{5-chloro-2-{(4-{[(4,5-dimethyl-1,3-thiazol-2-yl)amino]-methyl}phenyl)oxy}-3-pyridinyl}benzenesulfonamidetrifluoroacetate |
| 180 | 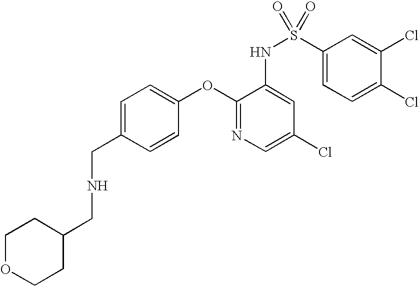 | 558 | 3,4-dichloro-N-{5-chloro-2-[(4-{[(tetrahydro-2H-pyran-4-ylmethyl)amino]-methyl}phenyl)oxy]-3-pyridinyl}benzenesulfonamidetrifluoroacetate |
| 181 | 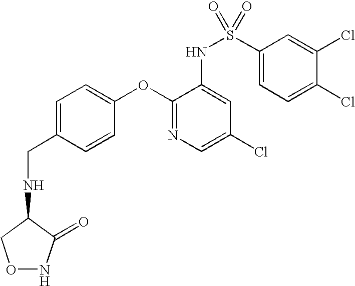 | 545 | 3,4-dichloro-N-(5-chloro-2-{[4-({[(4R)-3-oxo-4-isoxazolidinyl]amino}methyl)phenyl]oxy}-3-pyridinyl)benzenesulfonamidetrifluoroacetate |
| 182 | 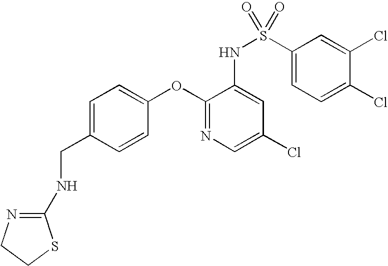 | 545 | 3,4-dichloro-N-[5-chloro-2-({4-[(4,5-dihydro-1,3-thiazol-2-ylamino)methyl]phenyl}oxy)-3-pyridinyl]benzenesulfonamidetrifluoroacetate |
| 183 | 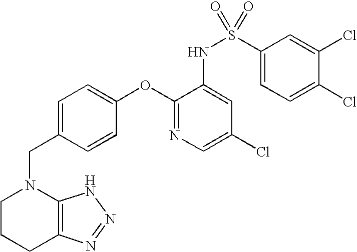 | 567 | 3,4-dichloro-N-(5-chloro-2-{[4-(1,5,6,7-tetrahydro-4H-[1,2,3]triazolo[4,5-b]pyridin-4-ylmethyl)phenyl]oxy}-3-pyridinyl)benzenesulfonamidetrifluoroacetate |
| 184 | 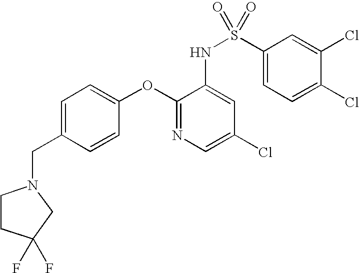 | 550 | 3,4-dichloro-N-[5-chloro-2-({4-[(3,3-difluoro-1-pyrrolidinyl)methyl]phenyl}oxy)-3-pyridinyl]benzenesulfonamidetrifluoroacetate |
| 185 | 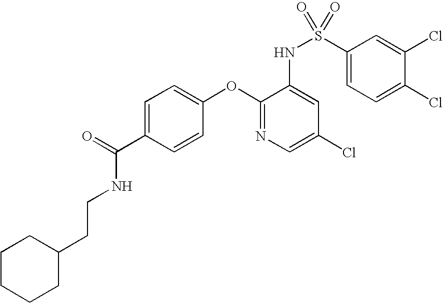 | 585 | 4-[(5-chloro-3-{[(3,4-dichlorophenyl)sulfonyl]amino}-2-pyridinyl)oxy]-N-[2-(1-piperidinyl)ethyl]benzamidetrifluoroacetate |
| 186 | 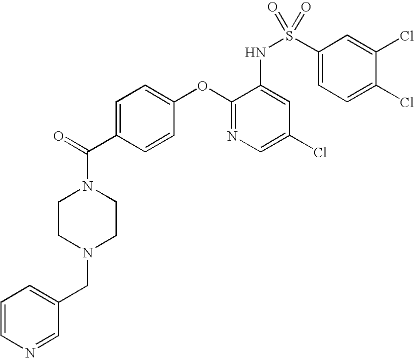 | 748 | 3,4-dichloro-N-{5-chloro-2-[(4-{[4-(3-pyridinylmethyl)-1-piperazinyl]carbonyl}phenyl)oxy}-3-pyridinyl}benzenesulfonamidetrifluoroacetate |
| 187 | 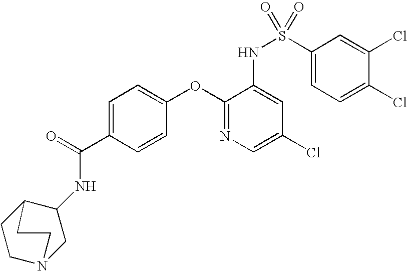 | 697 | N-1-azabicyclo[2.2.2]oct-3-yl-4-[(5-chloro-3-{[(3,4-dichlorophenyl)sulfonyl]amino}-2-pyridinyl)oxy]benzamide trifluoroacetate |
| 188 | 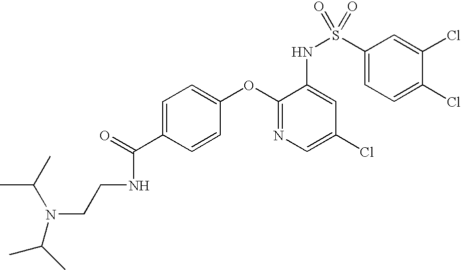 | 715 | N-{2-[bis(1-methylethyl)amino]ethyl}-4-[(5-chloro-3-{[(3,4-dichlorophenyl)sulfonyl]amino}-2-pyridinyl)oxy]benzamide trifluoroacetate |
| 189 | 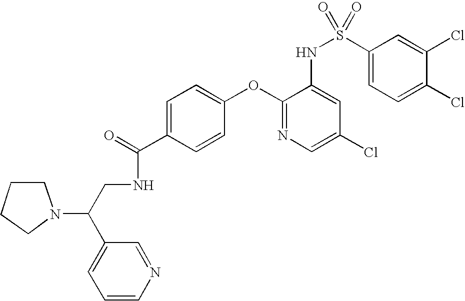 | 762 | 4-[(5-chloro-3-{[(3,4-dichlorophenyl)sulfonyl]amino}-2-pyridinyl)oxy]-N-[2-(3-pyridinyl)-2-(1-pyrrolidinyl)ethyl]benzamidetrifluoroacetate |
| 190 | 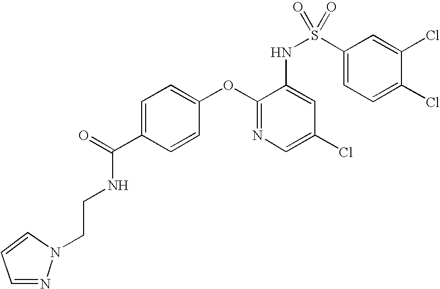 | 682 | 4-[(5-chloro-3-{[(3,4-dichlorophenyl)sulfonyl]amino}-2-pyridinyl)oxy]-N-[2-(1H-pyrazol-1-yl)ethyl]benzamide trifluoroacetate |

Compounds of the present invention have been found to exhibit affinity for chemokine receptors, in particular the CCR2 receptor. Such affinity is typically calculated from the IC₅₀ as the concentration of a compound necessary to inhibit 50% of the stimulated response from the receptor in an appropriate assay, and is reported as a “Kᵢ” value calculated by the following equation:

$${K}_{i} = \frac{{IC}_{50}}{1 + L / {K}_{D}}$$

where L=radioligand and K_{D}=affinity of radioligand for receptor (Cheng and Prusoff, Biochem. Pharmacol. 22:3099, 1973).

In the context of the present invention pKi (corresponding to the antilogarithm of Ki) is used instead of Ki.

In one aspect the present invention provides compounds having a pKi comprised between 5.4 and 6.5 as derived from the assay method set out below. In another aspect the present invention provides compounds having a pKi comprised between 6.5 and 7.0 as derived from such an assay. In a further aspect the present invention provides compounds having a pKi greater than 7.0 as derived from such an assay.

### CCR-2 [³⁵S] GTPgS SPA Binding Assay

### Membrane Preparation

CHO cells expressing the human CCR-2 receptor were grown in DMEM F12 media supplemented with 10% foetal calf serum, 2 mM L-glutamine, G418 at 37° C. 5% CO₂. Confluent cells were harvested using Hanks buffered salt solution (HBSS, Ca²⁺, Mg²⁺ free) containing 0.6 mM EDTA. The resulting cell suspension was centrifuged at 300 g at 4° C. for 10 min, cell pellet resuspended in 100 ml HBSS+EDTA and respun at 300 g for 5 min. The resulting cell pellet was resuspended in 50 mM HEPES containing 100 mM leupeptin, 25 μg/ml bacitracin, 1 mM EDTA, 1 mM PMSF and 2 μM pepstain A, at pH7.4. The suspension was homogenised using an ice cold blender and centrifuged at 500 g for 20 mins. The supernatant is withdrawn and spun at 48000 g for 30 mins. This cell pellet is resuspended in the above buffer minus the pepstatin A and PMSF and stored in aliquots at −70° C.

### Assay

For the assay, membranes are thawed and resuspended in assay buffer (20 mM HEPES, 10 mM MgCl₂, 100 mM NaCl, pH7.4, containing 1 mg/ml saponin, 10 mM GDP) to give final concentration of 5 μg/well. These are pre-coupled with LEADseeker SPA beads (0.25 mg/well) for 30 min at room temperature whilst mixing. Assay plates containing 0.5 μl of various test compounds (30 μM-30 pM) in 100% DMSO as 11 point, four fold dilutions across a 384 well plate are used in the assay which have been prepared on a Biomek FX. The plate also contains 16 wells of DMSO and 16 wells of a high concentration of a standard antagonist to produce high and low controls in the experiment. To this 15 μl of bead and membrane mix are added with, 15 μl [³⁵S] GTPgS (0.2 nM final assay concentration) and 15 μl of an EC₈₀ (40 nM) of MCP-1. This concentration of MCP-1 has been pre-determined from agonist curves run against this receptor. All additions are made using a multidrop. Plates are then sealed and centrifuged for 5 min at 300 rpm before they are left to incubate at room temperature for 3 hours. After this time they are read on a Viewlux imaging system. For data handling the high and low controls wells are used to normalise the data which is then fitted using a 4 parameter kit in Excel.

The assay described above is believed to have an effective limit of detection of a pKi in the region of 5.0-5.5. Accordingly, a compound exhibiting a pKi value within this range from such an assay may indeed have a reasonable affinity for the receptor, but equally it may also have a lower affinity, including a considerably lower affinity.

Using this assay, all of the exemplified compounds gave a pKi≧5.0.

1. A compound of the following formula: 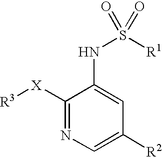
or a pharmaceutically acceptable salt thereof;
wherein:
X represents —O, —NH or —S;
R¹ is a heteroaryl group or an aryl group, each substituted with up to three substituents independently selected from the group consisting of halo, hydroxy, C₁₋₆ alkyl, C₁₋₄ alkoxy, hydroxy-C₁₋₄ alkyl-, C₁₋₄ alkoxy-C₁₋₄ alkyl-, C₃₋₆ cycloalkyl, —CN, C₁₋₄ alkylthio-, —OCF₃, dimethylamino, nitro, and —CF₃;
R² is H, C₁₋₆ alkyl, C₁₋₆ alkoxy, halo, —CN, —CF₃, or —OCF₃;
R³ is R⁴-phenyl-, R⁵-pyridyl-, methyltetrazolyl, morpholino-C(O)—CH₂—; (CH₃)₂N(CH₂)₂N(CH₃)—C(O)CH₂—, methylisoxazolyl; pyridazinyl, or triazolyl;
where R⁴ is H, halo, CN, hydroxymethyl, tetrazolyl, COOH, or —CR^{a}R^{b}—NRCR^{d};
R⁵ is 2-methyl, 2-halo, 2-cyano, 2-COOH, dimethylaminomethyl, methylmorpholinomethyl;
where R^{a} and R^{b} are each H or, together with the C atom to which they are attached, form a carbonyl group;
R^{c} is H, —(CH₂)_{y}—R⁶; C₃-C₆-cycloalkyl; C₁-C₆-alkyl; phenyl-CH(CH₃)—; oxoisoxazolidinyl; dimethylthiazolyl; dihydrothiazolyl; imidazolyl-CH₂CH(CH₂OH)—CH₂—; or imidazolyl-CH₂CH(CH₂OH)—;
R^{d} is H, C₁-C₄-alkyl, benzyl or, R^{c} and R^{d}, together with the nitrogen to which they are attached form a piperidinyl, pyrrolidinyl, difluoropyrrolidinyl, morpholino, pyrazinyl, triazolopiperidinyl, or pyrrolidinonyl group;
y is 1, 2, or 3;
R⁶=—NR⁷R⁸, OH, methoxy, phenyl, imidazolyl, indolyl, tetrahydropyranyl, benzimidazolyl, or C₃-C₆-cycloalkyl;
R⁷ is H or methyl; R⁸ is H, C₁-C₄-alkyl, or phenyl; or R⁷ and R⁸, together with the nitrogen to which they are attached form a piperidinyl, pyrrolidinyl, morpholino, pyrazinyl, imidazolyl, or pyrrolidinonyl group.

2. The compound of claim 1 or a pharmaceutically acceptable salt thereof wherein R² is Cl and X is O.

3. The compound of claim 2 or a pharmaceutically acceptable salt thereof wherein R¹ is a phenyl group with two substituents.

4. The compound of claim 3 which is represented by the following formula: 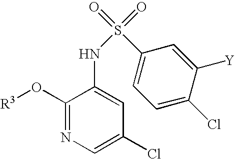
or a pharmaceutically acceptable salt thereof, where Y is C₁ or CF₃.

5. The compound of claim 4, or a pharmaceutically acceptable salt thereof, wherein R³ is R⁴-phenyl- or 2-methyl-3-pyridinyl.

6. The compound of claim 5, or a pharmaceutically acceptable salt thereof, wherein R⁴ is —CR^{a}R^{b}—NR^{c}R^{d}, wherein R^{c} is —(CH₂)_{y}—R⁶ and R^{d} is H.

7. The compound of claim 6 which is represented by the following formula: 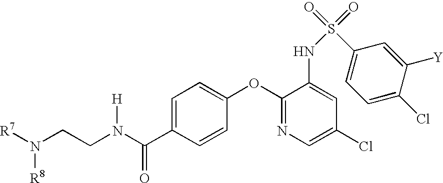
or a pharmaceutically acceptable salt thereof, wherein R⁷ and R⁸, together with the nitrogen to which they are attached form a piperidinyl, pyrrolidinyl, or morpholino, group.

8. A composition that comprises a) the compound of claim 1 or a pharmaceutically acceptable salt thereof, and b) a pharmaceutically acceptable excipient.

9. A method for treating or preventing a disease or condition mediated by CCR2 comprising administering to a patient in need thereof a pharmaceutically effective amount of the compound of claim 1, or a pharmaceutically acceptable salt thereof.
